(19) United States
(12) Patent Application Publication
(10) Pub. No.: US 2026/0077104 A1
(43) Pub. Date: Mar. 19, 2026

(54) Bioelectric Thread and Delivery Device

(71) Applicant: Brown University, Providence, RI (US)

(72) Inventors: Kareen L. K. Coulombe, Pawtucket, RI (US); Arvin H. Soepriatna, Providence, RI (US); Stephanie Roser, Providence, RI (US); Elena Song, Flower Mound, TX (US)

(21) Appl. No.: 19/334,764

(22) Filed: Sep. 19, 2025

Related U.S. Application Data
(60) Provisional application No. 63/696,725, filed on Sep. 19, 2024.

(51) Int. Cl. A61L 27/36; A61L 27/38

(52) U.S. Cl. CPC A61L 27/3625; A61L 27/367; A61L 27/3826; A61L 2430/20

(57) ABSTRACT

A method of formulating and delivering a bioelectric thread within tissue is provided by inserting a tube-loaded needle having the bioelectric thread contained therein within tissue, and removing the needle and tube thereby leaving the bioelectric thread within the tissue, wherein the bioelectric thread includes a crosslinked material substrate having electrically conductive cells, such as cardiomyocytes seeded thereon or within.

### CROSS-REFERENCE TO RELATED APPLICATIONS

This application claims benefit to U.S. provisional application 63/696,725 filed Sep. 19, 2024 and hereby incorporated by reference in its entirety.

### FEDERALLY SPONSORED RESEARCH AND DEVELOPMENT

This invention was made with government support under Grant Number 1 R21EB035030 awarded by the National Institutes of Health. The government has certain rights in the invention.

### TECHNICAL FIELD

The present disclosure relates to a substrate seeded or embedded with cells, such as cardiomyocytes, for the treatment of disease, such as cardiovascular disease, and to a method of delivering such a substrate seeded or embedded with cells within the human body, such as delivery to muscle tissue, in particular cardiac muscle tissue.

### DESCRIPTION OF THE RELATED ART

Cardiovascular disease (CVD) has been the leading cause of mortality in the United States for over a century, affecting approximately 48.6% of Americans older than 20 years of age. See S. S. Martin et al., Circulation, vol. 149, no. 8, pp. e347-e913, February 2024. A subset of these patients suffer from cardiac conduction disorders that often present as arrhythmias like bundle branch block or atrial fibrillation (AF). One in four adults over the age of 40 is likely to develop AF, and even healthy individuals without significant cardiac history have a lifetime risk of one in six. See D. M. Lloyd-Jones et al., Circulation, vol. 110, no. 9, pp. 1042-146 August 2004. Atrial fibrillation has been associated with 1.5 to 1.9-fold increases in risk of death as well as increased risk of stroke and heart failure. See E. J. Benjamin, P. A. et al., Circulation, vol. 98, no. 10, pp. 946-952, September 1998; S. Stewart, et al., Am. J. Med., vol. 113, no. 5, pp. 359-364, October 2002. Similarly, ventricular tachycardia (VT), another form of cardiac arrhythmia, is linked to higher incidences of sudden cardiac death. Heart attacks, or myocardial infarctions (MIs), are often responsible for acute coronary ischemia and resulting myocardial fibrosis which are common causes of arrhythmias including VT. See B. A. Koplan and W. G. Stevenson, Mayo Clin. Proc., vol. 84, no. 3, pp. 289-297, March 2009.

Fibrosis is a key contributor to the development of conduction disorders in all chambers of the heart. Following injury, the adult mammalian heart has limited regenerative capabilities despite the importance of repair mechanisms. In these processes, inflammatory cells infiltrate the ischemic tissue, necrotic cardiomyocytes (CMs) are cleared, and granulation tissue develops. See S. Verheule and U. Schotten, Cells, vol. 10, no. 11, p. 3220 November 2021. Quiescent cardiac fibroblasts (CFs) are activated into myofibroblasts that secrete contractile proteins and lead to the formation of collagen networks. See M. Liu, et al., Adv. Drug Deliv. Rev., vol. 173, pp. 504-519, June 2021. However, the surplus accumulation of extracellular matrix (ECM) proteins, particularly collagen I and III fibers, characterize fibrosis. Soon after injury, mature fibrotic scars form to maintain the myocardium's structural integrity. While initial ECM deposition is necessary for wound healing and moderating cell proliferation and differentiation, prolonged and excessive tissue damage stimulates continuous ECM deposition changing organ architecture, causing cardiac dysrhythmias, and impairing cardiac function. Sec K. Maruyama and K. Imanaka-Yoshida, Int. J. Mol. Sci., vol. 23, no. 5, Art. no. 5, January 2022.

In healthy tissues, cardiac action potentials travel through gap junction channels between adjacent CMs and and between CMs and CFs. In mature CMs, large gap junctions form at intercalated discs in end-to-end connections while smaller junctions are observed in side-to-side connections. The latter transverse connections are influenced by the presence of endomysial and fibrous tissue. When fibrous scar tissue forms, the collagenous septa between the CMs expand, electrically isolating CMs and reducing their electrical coupling. Sec C. Sohns and N. F. Marrouche, Eur. Heart J., vol. 41, no. 10, pp. 1123-1131 March 2020. Electrical propagation must then form alternative paths, slowing propagation or conduction velocity. Additionally, in diseased hearts, connexin 43 (Cx43), the predominant cardiac gap junction protein, is downregulated, further decreasing conduction velocity along the major axis of the fiber direction. See T. P. Nguyen, Z. Qu, and J. N. Weiss, J. Mol. Cell. Cardiol., vol. 70, pp. 83-91, May 2014. Meanwhile, it has been suggested that CM-CF coupling changes in disease and can induce spontaneous CM depolarization and shorten action potentials, increasing the likelihood of ectopic impulses and reentrant arrhythmias. Sec S. Nattel, JACC Clin. Electrophysiol., vol. 3, no. 5, pp. 425-435, May 2017. Other clinical conditions such as atrioventricular block, PR prolongation, and left and right bundle branch blocks have also been attributed to fibrosis of the cardiac conduction system in adult or congenital heart disease. See T. Kerola et al., JAMA Netw. Open, vol. 2, no. 5, p. e194176, May 2019.

Current therapies for cardiac conduction disorders include the use of pacemakers, implantable cardioverter defibrillators (ICDs), ablation procedures, and medications. While cardiac resynchronization therapy through the implantation of a pacemaker device has been effective in counteracting the consequences of left ventricular dysfunction and dyssynchrony, 10-15% of implantation procedures are met with complications. See M. Fatemi, et al., EP Eur., vol. 10, no. 11, pp. 1308-1312 November 2008. Pacemaker infection, device erosion, pneumothoraces, thrombosis, and invasive follow up procedures are common risks for patients with permanent pacemaker therapy. See M. S. Kiviniemi, et al., Pacing Clin. Electrophysiol., vol. 22, no. 5, pp. 711-720, 1999. These major complications contribute a non-negligible financial burden to healthcare systems. See N. Clémenty et al., J. Med. Econ., vol. 22, no. 11, pp. 1171-1178 November 2019.

ICDs may be recommended for patients with risks for sudden cardiac death due to dangerous arrhythmias like ventricular tachycardia or ventricular fibrillation. See A. F. L. Schinkel, et al., Circ. Heart Fail., vol. 5, no. 5, pp. 552-559, September 2012. However, these devices pose similar issues and consequences to pacemakers. In a study of 440 ICD recipients, 31% experienced complications with 12% receiving inappropriate shocks during a mean follow-up period of 46 months. See P. Alter, et al., Pacing Clin. Electrophysiol. PACE, vol. 28, no. 9, pp. 926-932, September 2005. Catheter ablation procedures are often conducted for patients who are unresponsive to pharmacological therapies. In cases of both atrial and ventricular arrhythmias, procedural complications were seen with vascular access in addition to thrombosis, nerve damage, cardiac tamponade, and valvular damage. See A. Gupta et al., Circ. Arrhythm. Electrophysiol., vol. 6, no. 6, pp. 1082-188 December 2013. Furthermore, only 25% of first-attempt ablations are successful and only 68% of multiple-procedure attempts have positive outcomes. See S. Brooks et al., J. Cardiovasc. Electrophysiol., vol. 29, no. 2, pp. 257-263, February 2018. Foreign body devices and procedural interventions provide limitations demonstrating a need for a more reliable treatment for cardiac conduction disorders.

Human induced pluripotent stem cells (hiPSCs) are a renewable source of CMs for cardiovascular regenerative therapies, and recent advances have strengthened the understanding of their biology. See M. A. Laflamme and C. E. Murry, Nature, vol. 473, no. 7347, pp. 326-335, May 2011. Furthermore, induced pluripotent stem cells have been shown to be engineered to reduce their immunogenicity, allowing recipients to avoid immunosuppression and demonstrating the potential for universal acceptance. See T. Deuse et al., Nat. Biotechnol., vol. 37, no. 3, pp. 252-258, March 2019. Robust and reliable protocols have been optimized for the differentiation of atrial and ventricular phenotypes of human induced pluripotent stem cell-derived cardiomyocytes (hiPSC-CMs) with >70%-95% purity through temporal modulation of the Wnt signaling pathway. See C. E. Rupert, C. Irofuala, and K. L. K. Coulombe, PLOS ONE, vol. 15, no. 3, p. e0230001, March 2020. However, the field of heart regeneration with engineered cardiac tissues is still limited by the lack of electrical coupling between hiPSC-CMs implanted on the epicardial surface and host cardiomyocytes. See K. A. Gerbin, X. Yang, C. E. Murry, and K. L. K. Coulombe, PLOS ONE, vol. 10, no. 7, p. e0131446, July 2015.

Attempts have been made to inject a continuous stream of human embryonic stem cell-derived cardiomyocytes into an infarcted myocardium; nevertheless, electrical syncytium between these CMs was not achieved prior to implantation in this study, relying on maturation and formation of gap junctions in vivo. See J. Luo, et al., J. Thorac. Cardiovasc. Surg., vol. 148, no. 6, pp. 3180-3188.e1, December 2014. Yet, other work has shown that a suspension of pluripotent stem cell-derived CMs can create transient ventricular arrhythmias when injected into animal models due to immature phenotypes serving as ectopic pacemakers. See S. Kadota, et al., J. Cardiol., vol. 76, no. 5, pp. 459-463, November 2020.

Needle injection-based delivery systems of stem-cell derived cardiomyocytes have been commonly used for in vivo studies as well as clinical trials (e.g., NCT04396899), but successful engraftment of these cell suspensions has been severely limited due to wash out and cell death. See R. Tabei et al., J. Heart Lung Transplant. Off. Publ. Int. Soc. Heart Transplant., vol. 38, no. 2, pp. 203-214, February 2019. Conversely, functional cardiac patches have been delivered using a minimally invasive injection, without harming CM survival or function. See M. Montgomery et al., Nat. Mater., vol. 16, no. 10, pp. 1038-146 October 2017. Meanwhile, other fields of healthcare and cosmetology also often use injection-based delivery systems. Notably, the thread-lift, an increasingly popular cosmetic procedure, uses a needle to insert degradable barbed polymer suture subcutaneously. See S. Eremia and M. A. Willoughby, Dermatol. Surg., vol. 32, no. 3, p. 335, March 2006.

Taken together, there is an urgent need for biomedical innovations to reengineer physiological electrical conduction pathways, such as in diseased hearts. There is an additional need for delivering an implant including a substrate having cells adhered thereto into muscle tissue in need thereof.

### SUMMARY OF THE INVENTION

The present disclosure provides in general, an electrically conductive biological device including electrical impulse transmitting cells and a substrate supporting the electrical impulse transmitting cells. The present disclosure provides an electrically conductive biological device including a combination of a substrate and cells, such as electrical impulse transmitting cells, an example of which includes cardiomyocytes. According to one exemplary aspect, the combination of the substrate and electrical impulse transmitting cells can be used to reengineer conduction pathways in tissue in need thereof by providing an electrical function promoting electrical coupling capability. According to one exemplary aspect, the combination of the substrate and cardiomyocytes can be used to reengineer cardiac conduction pathways by providing an electrical function promoting electrical coupling capability. The combination providing electrical function when administered on or into tissue, such as muscle tissue, is referred to herein as a “bioelectric thread.” Bioelectric threads comprising electrical impulse transmitting cells, such as cardiomyocytes exhibit a functioning electrical syncytium, i.e. a network of electrically connected cells that function as a single unit, allowing for the rapid, coordinated spread of electrical signals like action potentials. In the bioelectric threads described herein, individual cells are joined by gap junctions, enabling the quick transfer of ions and excitation from one cell to the next.

The combination of a substrate and electrical impulse transmitting cells can be delivered to tissue, such as muscle tissue, within a patient using methods described herein. According to one aspect, the substrate can be seeded with electrical impulse transmitting cells on the surface thereof. According to one aspect, the substrate can be seeded with electrical impulse transmitting cells embedded within the substrate as a matrix to support the electrical impulse transmitting cells. The combination of a substrate and electrical impulse transmitting cells can be used in the treatment of a patient in need of such treatment, for the treatment of disease, such as cardiovascular disease, and to a method of delivering such a combination of a substrate and electrical impulse transmitting cells within the human body, such as delivery to muscle tissue, in particular cardiac muscle tissue. According to one aspect, the present disclosure provides methods and devices for the delivery of a bioelectric thread including cardiomyocytes to the heart to create an electrically conductive cell bridge to improve coordination of cardiac excitation and/or the regeneration of conduction in multiple etiologies of conduction disorder in the heart with the long-term goal of reducing arrhythmia, establishing normal heart rhythm and improving patient outcomes.

According to one aspect, a bioelectric thread is described herein to be used to provide an electrical bridge to treat conduction disorders by bridging a failure of conduction between local islands of tissue that are separated physically by millimeter (mm) to centimeter (cm) distances of non-conductive or poorly conductive tissue. According to one aspect, a bioelectric thread is described herein to be used to provide an electrical bridge to treat conduction disorders by bridging a failure of conduction between local islands of cardiomyocytes that are separated physically by millimeter (mm) to centimeter (cm) distances of non-conductive or poorly conductive tissue. According to one exemplary aspect, the bioelectric threads described herein provide cell-based physical connections between electrically active cardiomyocytes that resynchronize cardiac electrophysiology to reduce arrhythmia risk and promote efficient cardiac pumping of blood. According to one aspect, the bioelectric thread includes a natural biomaterial and electrical impulse transmitting cells. According to one aspect, the bioelectric thread includes a natural biomaterial and cardiomyocytes, such as hiPSC-derived cardiomyocytes (hiPSC-CMs). According to one aspect, the bioelectric thread includes a natural biomaterial fiber contacted with or otherwise coated with or embedded with electrical impulse transmitting cells, such as cardiomyocytes or hiPSC-derived cardiomyocytes (hiPSC-CMs).

According to one aspect, the bioelectric thread includes electrical impulse transmitting cells dispersed or embedded within a natural biomaterial, which may be in the form of a thread or fiber (or other form) as described herein. According to this aspect, a natural biomaterial and electrical impulse transmitting cells may be mixed together and a substrate of the mixture may be formed and processed as described herein.

According to one aspect, the bioelectric thread includes a core-shell embodiment where a natural biomaterial core is provided with a shell of a matrix material. Electrical impulse transmitting cells may be within the core of the natural biomaterial. Electrical impulse transmitting cells may be within the shell matrix material, which may be a natural biomaterial. Electrical impulse transmitting cells may be within both the core and the shell matrix material. Core-shell embodiments can be produced using core-shell co-extrusion methods known to those of skill in the art using biomaterials alone or mixed with electrical impulse transmitting cells as described herein.

According to one aspect, the electrically conductive biological device, such as a bioelectric thread, provides electrical impulse transmitting cell-based coupling across mm to cm distances via formation of a continuous bridge of electrical impulse transmitting cells, such as hiPSC-CMs. The delivery of confluent layers or connections of electrical impulse transmitting cells such as cardiomyocytes along the surface of or embedded within threads as described herein creates an electrical bridge via cellular gap junctions with known conduction velocity. Exemplary hiPSC-CM conduction along the surface of or embedded within threads as described herein transmits action potential signals and calcium transients across at least 1.5 cm at 2.7 cm/s conduction velocity between two engineered cardiac tissues. According to one aspect, methods and devices are provided to establish electrical connections across defective conduction regions to restore impulse propagation. According to one aspect, methods and devices are provided to establish electrical connections across defective conduction regions to restore impulse propagation and resynchronize cardiac contraction to reduce arrhythmia risk and promote efficient cardiac contraction.

According to certain embodiments, methods are provided to optimize delivery of bioelectric threads into muscle tissue by increasing the mechanical integrity and handleability of the thread through crosslinking of a substrate with (1) gelation or a crosslinker, (2) by reducing shear of electrical impulse transmitting cells, such as cardiomyocyte cells, in or on the bioelectric thread using a friction reducing material such as a lubricant or surface modification or treatment of delivery devices to reduce friction during the delivery method, (3) by using a split sheath embodiment to assist with delivery of the bioelectric thread using an injection-based method into the muscle tissue, and/or (4) by using a needle including two longitudinal, interlocking, and separable portions between which the electrically conductive biological device is positioned thereby preserving electrical impulse transmitting cells at the surface of the bioelectric thread and providing electrical function of the bioelectrical thread implanted within muscle tissue.

### BRIEF DESCRIPTION OF THE DRA WINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

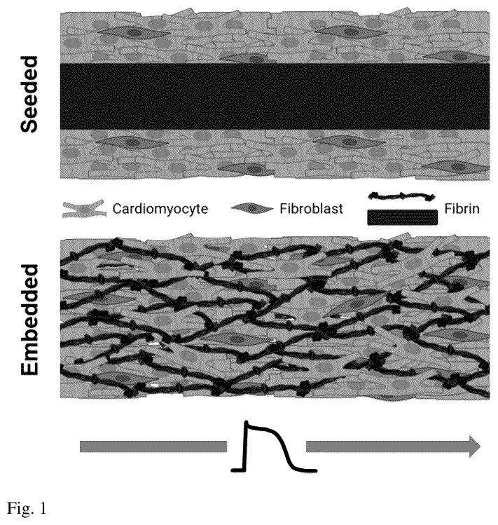

FIG. 1 is a schematic of a bioelectric thread as described herein wherein a fibrin thread is either coated in (top) or embedded with (bottom) hiPSC-CMs, and action potentials are able to propagate from one end to the other.

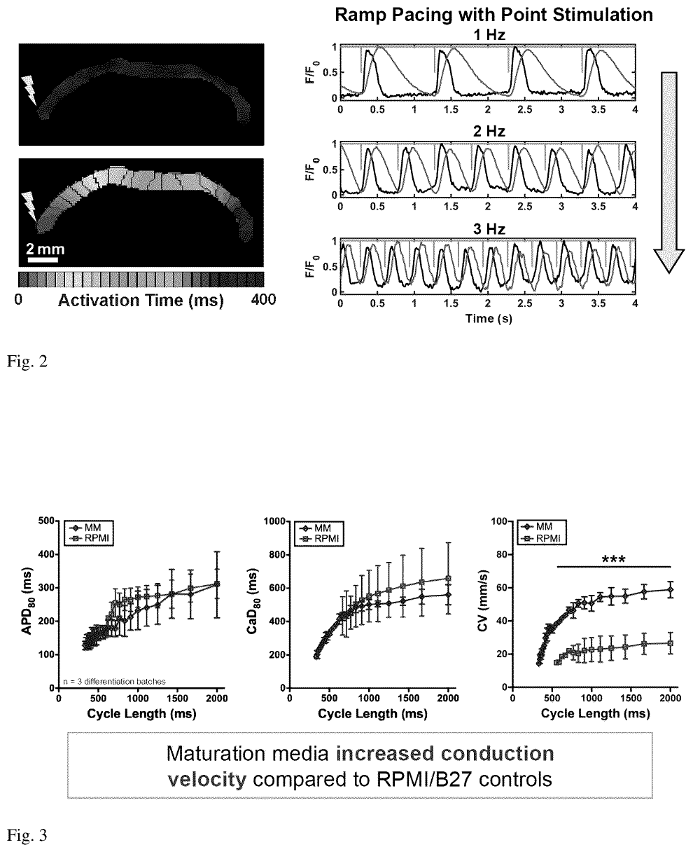

FIG. 2 shows that applying point electrical stimulation at one end of the bioelectric thread enabled uniform propagation of electrical signal to the opposite end, up to a frequency of 3 Hz or 180 beats per minute, at the upper end of the range of human heart rates during exercise.

FIG. 3 depicts that bioelectric threads exhibit frequency-dependent electrophysiological responses, including action potential duration (APD) and calcium duration (CaD) shortening and reductions in conduction velocities (CV) at shorter cycle length. Threads cultured in maturation media exhibit significantly faster CVs compared to threads cultured in standard RPMI media. N=3 differentiation batches.

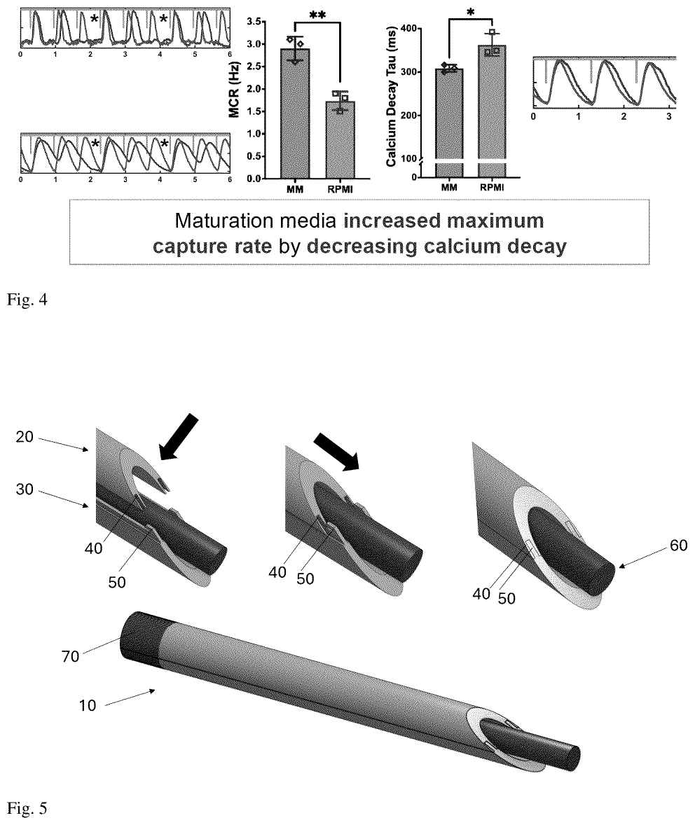

FIG. 4 depicts that bioelectric threads cultured in maturation media achieved significantly higher maximum capture rates than threads cultured in standard RPMI media by decreasing calcium decay tau.

FIG. 5 depicts in schematic an embodiment of a needle including two longitudinal, interlocking, and separable portions between which the bioelectric thread is positioned.

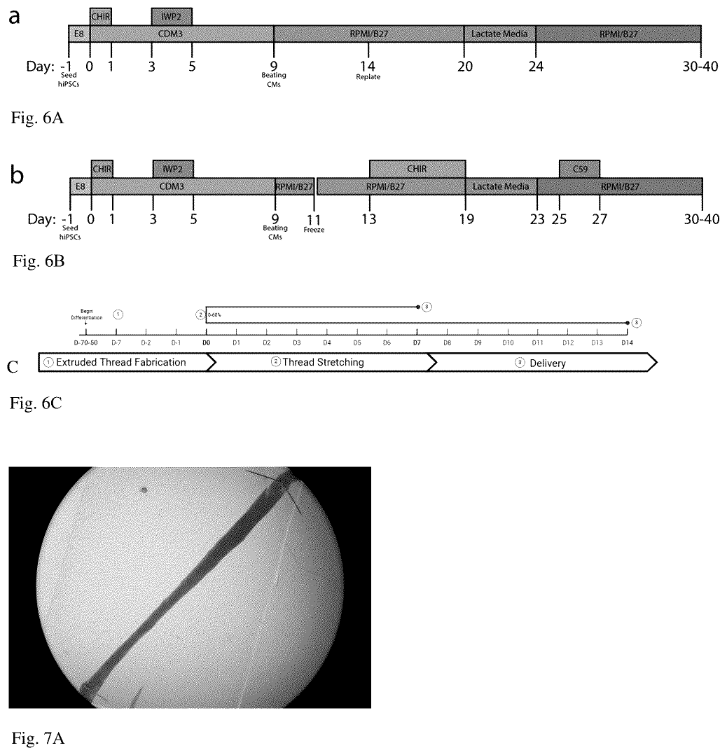

FIG. 6A is a schematic depicting a non-expanded differentiation process.

FIG. 6B is a schematic depicting an expanded differentiation process with a freeze/thaw step.

FIG. 6C is a schematic depicting an extruded thread fabrication process followed by thread stretching and delivery.

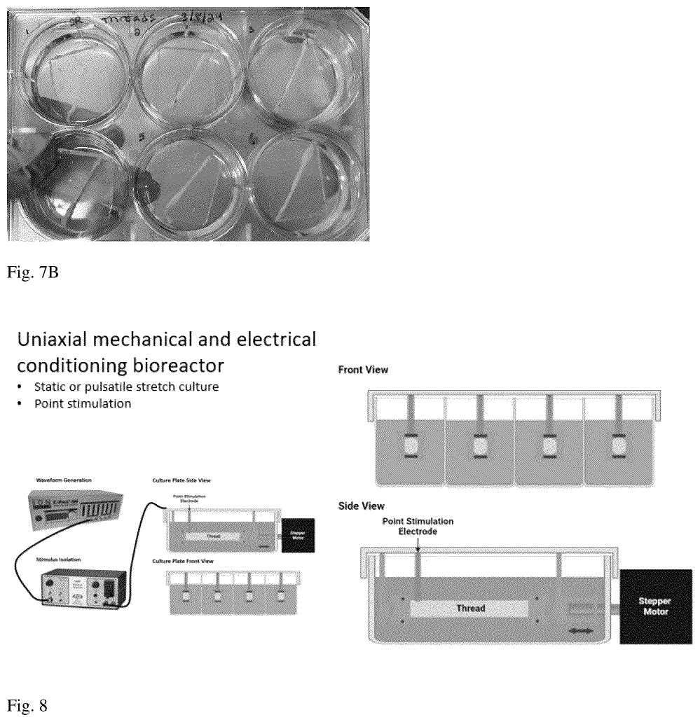

FIGS. 7A and 7B depict representative bioelectric threads with embedded cardiomyocytes produced by the methods described herein.

FIG. 8 illustrates a uniaxial mechanical and electrical conditioning bioreactor that can be used in the bioelectric thread manufacturing methods described herein.

FIG. 9 shows scanning electron microscope images showing surface level cell alignment of cells in a bioelectric thread produced by the methods described herein, with the black dashed line indicating the axis of applied tension.

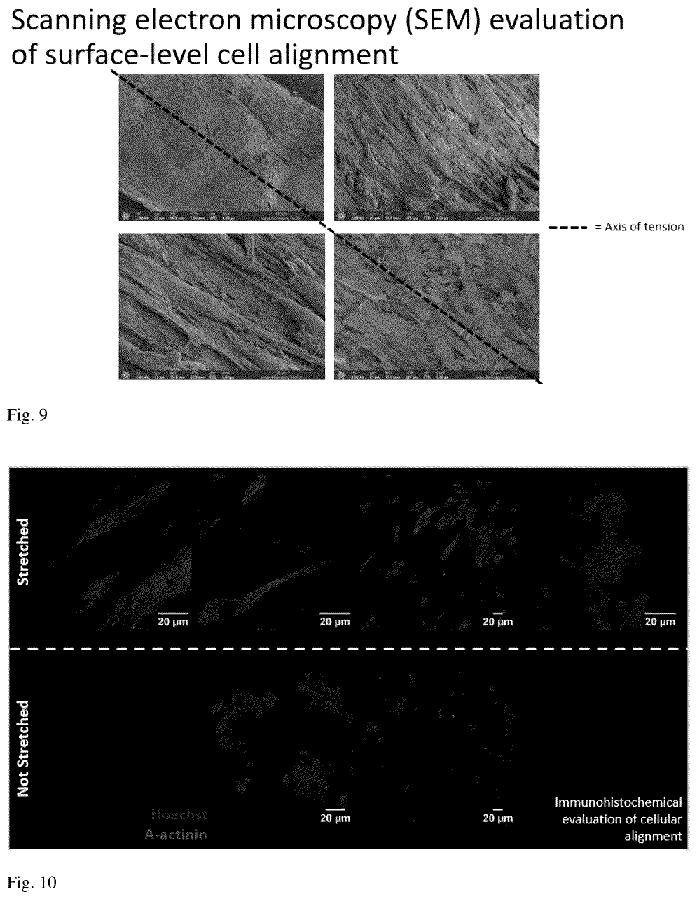

FIG. 10 are immunohistochemical images with actin filaments labeled in red and cell nuclei labeled in blue demonstrating cellular alignment of stretched versus unstretched bioelectric threads produced by the methods described herein.

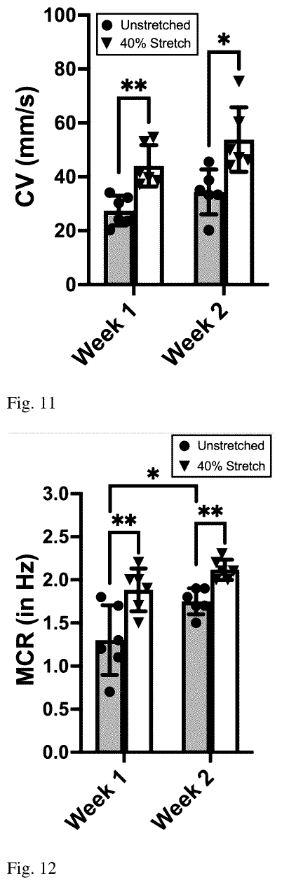

FIG. 11 depicts that stretched bioelectric threads significantly increased conduction velocity compared (CV) to unstretched threads.

FIG. 12 depicts that stretched bioelectric threads significantly increased maximum capture rates (MCR) compared to unstretched threads.

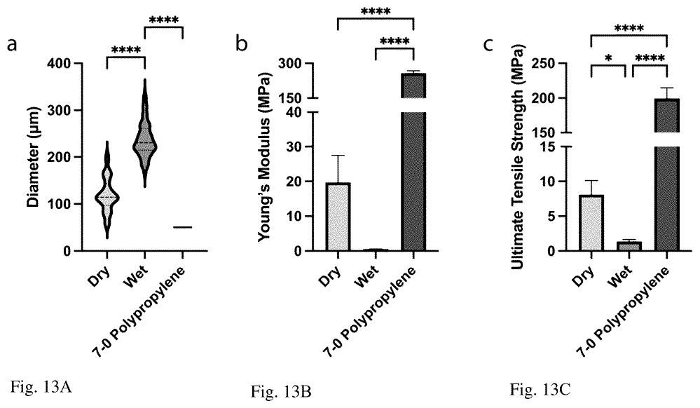

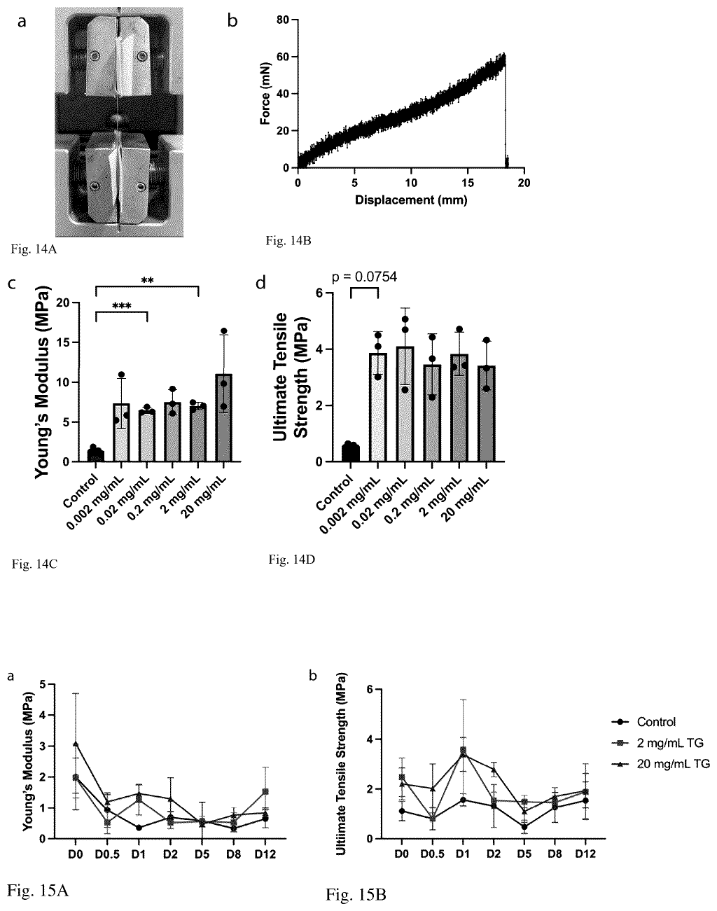

FIG. 13A depicts comparative data of the diameter of dry fibrin threads, wet fibrin threads and 7-0 polypropylene suture.

FIG. 13B depicts comparative data of Young's modulus (MPa) for dry fibrin threads, wet fibrin threads and 7-0 polypropylene suture.

FIG. 13C depicts comparative data for ultimate tensile strength for dry fibrin threads, wet fibrin threads and 7-0 polypropylene suture.

FIG. 14A depicts a tensile strength testing setup.

FIG. 14B is a graph of representative force (mN) vs. displacement (mm) during tensile testing.

FIG. 14C depicts data of Young's modulus calculated as the slope of the linear region of the stress-strain curve produced by normalizing force and displacement to the initial sample cross sectional area and gauge length, respectively.

FIG. 14D depicts data of ultimate tensile strength defined as the maximum stress on the specimen before failure (n=3 for all conditions).

FIG. 15A is a graph of Young's modulus (MPa) of uncrosslinked and crosslinked threads from Day 0 to Day 12.

FIG. 15B is a graph of ultimate tensile strength (mPa) of uncrosslinked and crosslinked threads from Day 0 to Day 12.

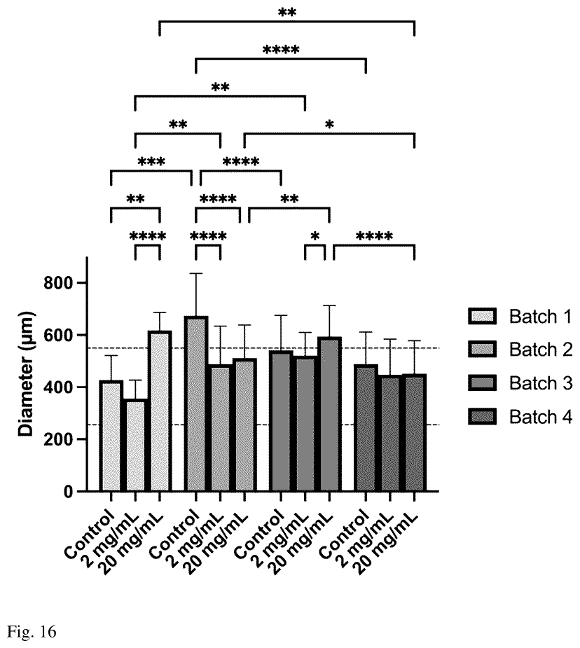

FIG. 16 depicts bioelectric thread diameters, measured using image analysis via ImageJ, of four fabrication batches of threads crosslinked using 0, 2, or 20 mg/mL transglutaminase.

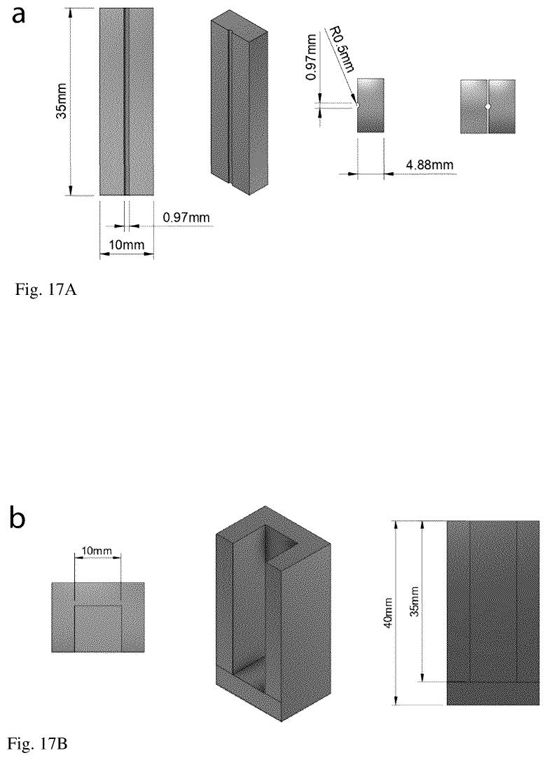

FIG. 17A depicts in schematic two 35 mm blocks with a channel cut down the middle of each block to accommodate a polymer tube, such as polyurethane, polylropylene, polyvinylchloride and the like. The two blocks are the placed together to enclose a polymer tube.

FIG. 17B depicts a holder for holding the two blocks placed together.

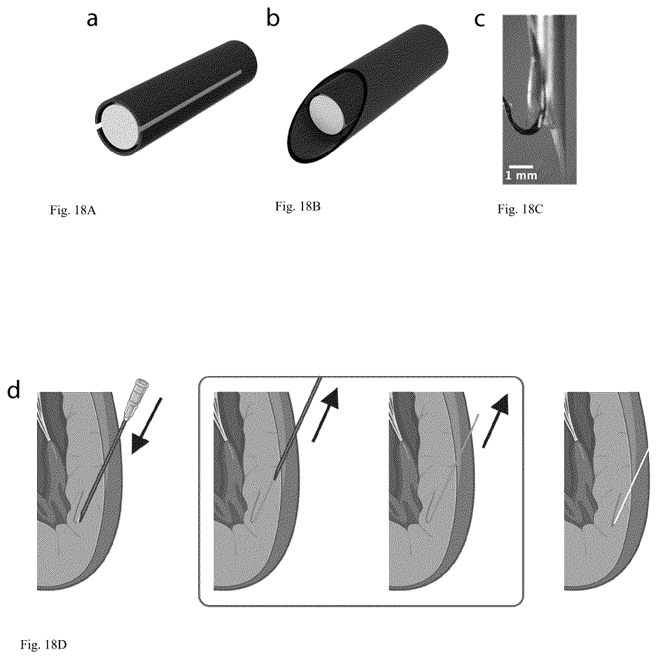

FIG. 18A is a schematic of a bioelectric thread placed into sliced tubing.

FIG. 18B is a schematic of the thread-containing tubing loaded into an 18 gauge needle.

FIG. 18C is an image of the delivery device (thread within sliced tubing within a needle) with the acellular thread having an exposed tail portion outside of the sliced tubing. The thread was artificially colored blue to be visible in the image of the delivery device.

FIG. 18D is a schematic of the needle with the sliced tubing therein and with the bioelectric thread within the sliced tubing injected into the host tissue. The needle is removed. Then the sliced tubing is removed leaving the bioelectric thread within the host muscle tissue.

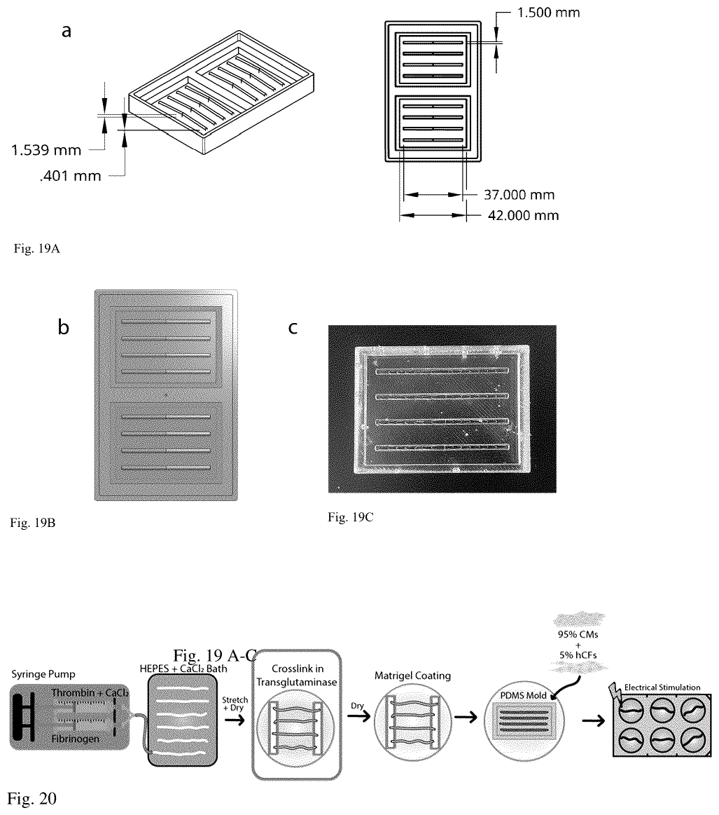

FIG. 19A depicts illustrations and dimensions of a cell-seeding mold.

FIG. 19B depicts a rendering of a negative PDMS mold.

FIG. 19C depicts a PDMS cell-seeding mold made from a negative PDMS mold.

FIG. 20 depicts in schematic a method for bioelectric thread fabrication.

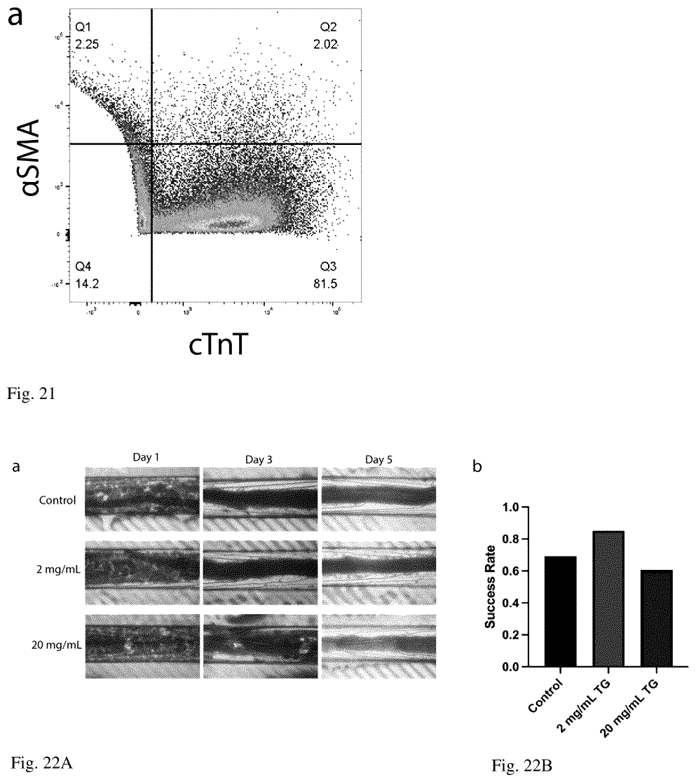

FIG. 21 depicts representative flow cytometry data demonstrating greater then 70% (81.5%) purity of hiPSC-CMs.

FIG. 22A shows representative images of bioelectric threads and cell compaction on threads over time in seeding molds.

FIG. 22B depicts comparative success rate data of the control, 2 mg/ml transglutaminase crosslinked thread and the 20 mg/ml transglutaminase crosslinked thread.

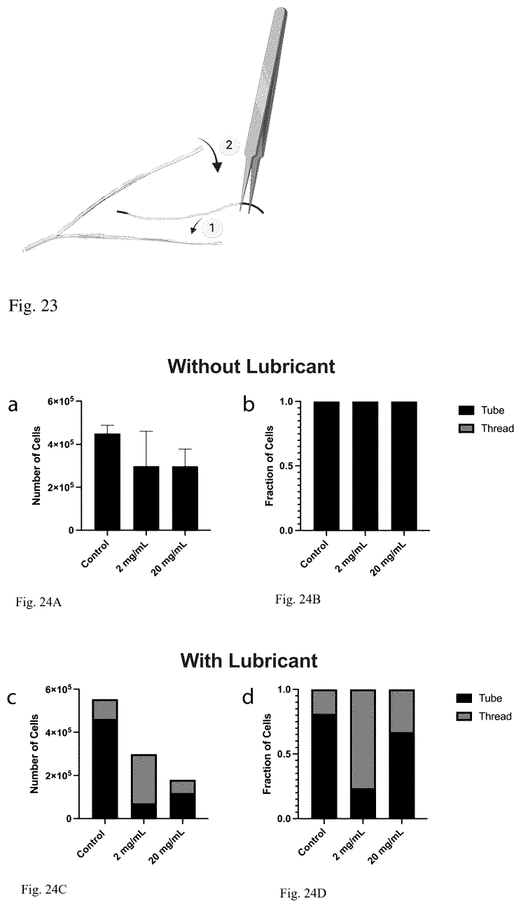

FIG. 23 depicts in schematic loading of the bioelectric thread into the sliced tubing with a portion of the thread without cell seeding exposed from the sliced tubing. Two halves of the tube are shown in an open position. Forceps are used to place a bioelectric thread onto the lower half of the tube in a first step 1 with a portion of the bioelectric thread extending outward from the tube. In a second step 2, the top half is positioned on the lower half in a closed position (not shown).

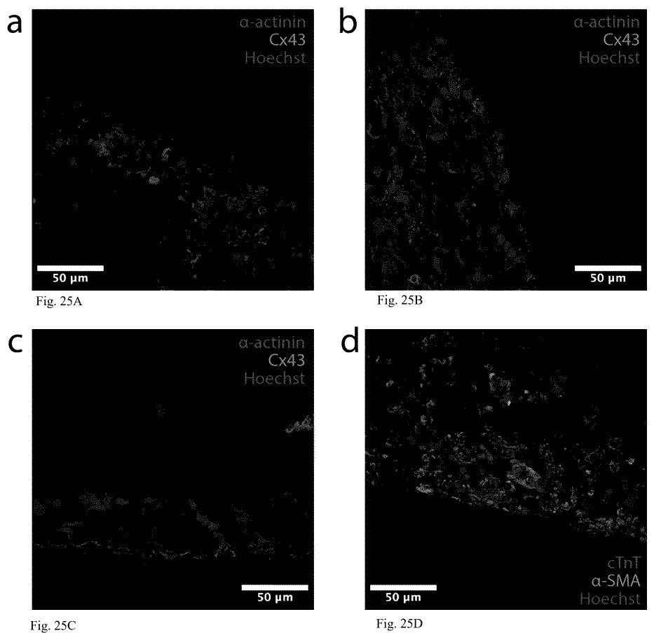

FIG. 24A depicts the number of cells remaining in the tube (black) versus delivered with the thread (pink) at varying cross-linking conditions without using lubricant.

FIG. 24B depicts the calculated fraction of cells remaining in the tube (black) or successfully delivered with the thread (pink) without using lubricant.

FIG. 24C depicts the number of cells remaining in the tube (black) versus delivered with the thread (pink) at varying cross-linking conditions when using lubricant.

FIG. 24D depicts the calculated fraction of cells remaining in the tube (black) or successfully delivered with the thread (pink) without using lubricant.

FIG. 25A depicts immunohistological staining of a control thread against alpha-actinin and connexin 43 and counterstained for nuclei.

FIG. 25B depicts immunohistological staining of a 2 mg/mL crosslinked thread against alpha-actinin and connexin 43 and counterstained for nuclei.

FIG. 25C depicts immunohistological staining of a 20 mg/mL crosslinked thread against alpha-actinin and connexin 43 and counterstained for nuclei.

FIG. 25D depicts immunohistological staining of a control thread against cardiac troponin T and alpha-smooth muscle actin with nuclear counterstain.

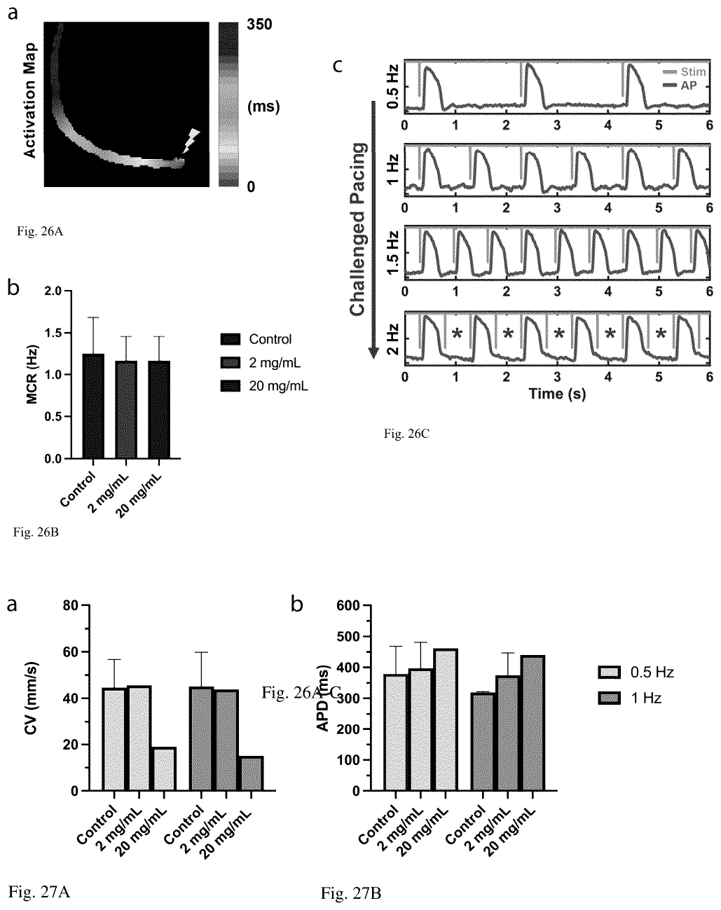

FIG. 26A depicts an activation map of action potential propagation along a representative bioelectric thread, with the yellow bolt indicating the point stimulation location.

FIG. 26B depicts data suggesting that no significant differences were seen in maximum capture rate with transglutaminase crosslinking (Control n=3, 2 mg/mL n=3, 20 mg/mL n=3).

FIG. 26C depicts representative action potential traces as frequency of electrical pacing increased with red asterisks indicating missed beats.

FIG. 27A depicts conduction velocity under different concentrations of transglutaminase crosslinking (Control n=3, 2 mg/mL n=1, 20 mg/mL n=1).

FIG. 27B depicts maximum rate repolarization action potential duration with varying concentrations of transglutaminase crosslinking (Control n=3, 2 mg/mL n=3, 20 mg/mL n=1).

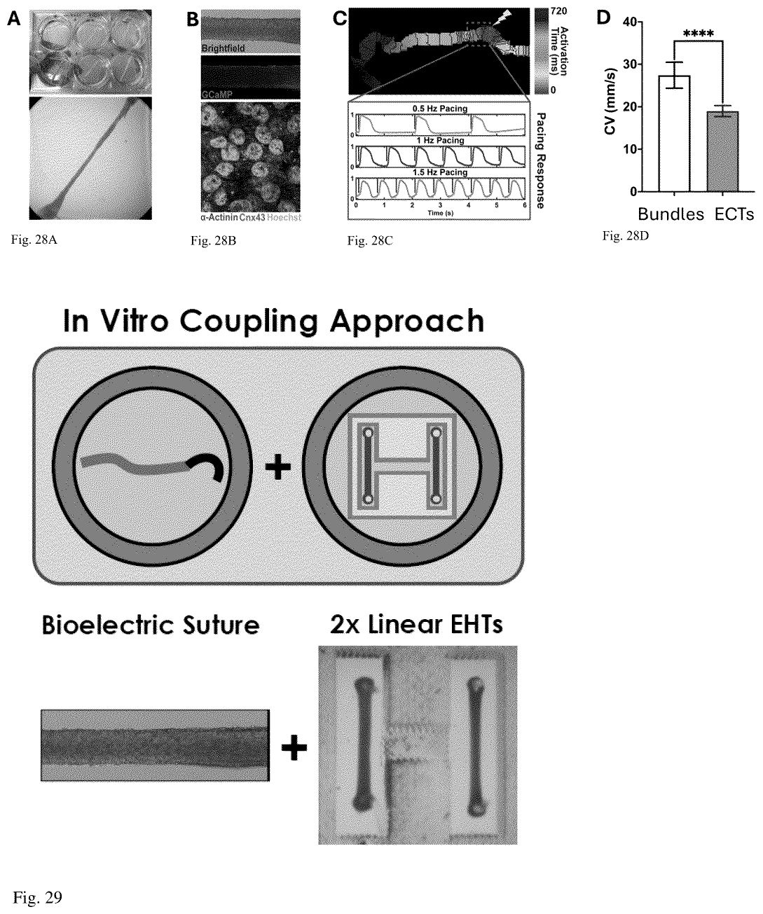

FIG. 28A is an image depicting needle-extruded bundles of ventricular hiPSC-CMs in a fibrinogen-gelatin hydrogel pinned onto PDMS substrates to apply static stretch.

FIG. 28B is an image depicting visible uniform cells with CMs having active GCaMP calcium signal, sarcomere organization, and gap junctions.

FIG. 28C depicts optical mapping results showing propagation of voltage from a point stimulus (lightning bolt) in a free-floating fiber (no tension) with physiological frequency response.

FIG. 28D depicts data showing that bioelectric threads (labeled bundles) exhibit faster conduction velocity (CV) than engineered cardiac tissues (ECTs) from the same batch of hiPSC-CMs.

FIG. 29 provides an experimental schematic for in vitro coupling of bioelectric threads with two linear engineered cardiac tissues (ECTs).

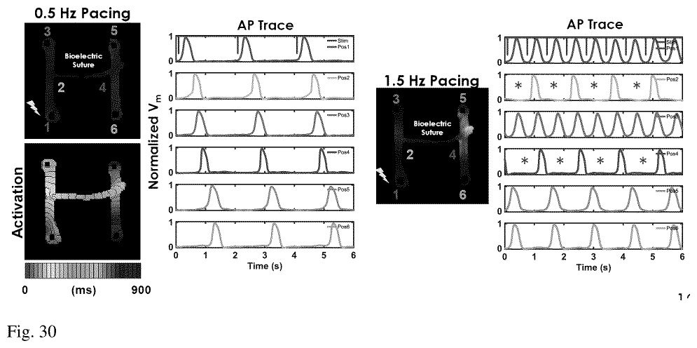

FIG. 30 demonstrates that bioelectric threads electrically couple in vitro with two engineered cardiac tissues (ECTs) within 1 day, directing electrical signal propagation from the left ECT to right ECT via the thread.

### DETAILED DESCRIPTION

The present disclosure provides an electrically conductive biological device including a biocompatible support material, and electrical impulse transmitting cells supported by the biocompatible support material wherein the electrical impulse transmitting cells are in the form of a network of electrically conductive electrical impulse transmitting cells. The electrically conductive biological device may be in the form of a thread, suture, string, fiber, strip or patch. Aspects of the present disclosure are directed to a substrate, such as a substrate of biological material, and electrical impulse transmitting cells using the substrate as a support referred to herein as a “bioelectric thread.” According to one aspect, the substrate is in the form of a thread and electrical impulse transmitting cells, such as cardiomyocytes are contacted with, are attached to, are embedded on or in, or are otherwise seeded on or in the substrate. According to one aspect, cardiomyocytes seeded or embedded on or in the substrate exhibit electrical behavior similar to native healthy cardiomyocytes found in cardiac muscle tissue. The bioelectric thread is delivered or otherwise administered to cardiac tissue ex vivo, in vitro or in vivo using an injection-based delivery device and method where the bioelectric thread is contained within a needle and the needle is inserted into muscle tissue and the needle is withdrawn leaving the bioelectric thread inserted into the interior of the cardiac tissue. According to one aspect, the bioelectric thread may be within a tube which is within the needle, and the needle may be withdrawn leaving the tube with the bioelectric thread therein within the tissue. The tube may then be withdrawn leaving the bioelectric thread within the tissue. According to one aspect, the needle and the tube may be withdrawn simultaneously, thereby leaving the bioelectric thread within the tissue. Aspects of the present disclosure address making the substrate and seeding or embedding with electrical impulse transmitting cells, such as cardiomyocytes. Aspects of the present disclosure address making a mixture of substrate material and electrical impulse transmitting cells such as cardiomyocytes and forming a thread or fiber from the mixture. Aspects of the present disclosure address making a core-shell structure of a core of biomaterial and a shell of a matrix material, with electrical impulse transmitting cells, such as cardiomyocytes, in the core or the shell or both, using a core-shell co-extrusion process. Each of these embodiments may be referred to herein as a “bioelectric thread.” Aspects of the present disclosure address delivering the bioelectric thread to muscle tissue, such as cardiac muscle tissue. Aspects of the present disclosure address providing electrical function of the bioelectric thread to muscle tissue, such as cardiac muscle tissue.

Aspects of the present disclosure contemplate sutures also referred to herein as threads or fibers, made of biological materials, like collagen, gelatin, fibrin, chitosan, alginate, hyaluronic and the like or mixtures thereof, and including electrical impulse transmitting cells, such as cardiomyocytes for use in cardiac tissue engineering. According to one aspect, the material of the suture or thread or fiber or other structure described herein is crosslinked to increase the mechanical properties of the suture or thread or fiber or other structure described herein.

According to one aspect, a bioelectric thread as described herein facilitates a delivery of electrical impulse transmitting cells, such as cardiomyocytes, to the host muscle tissue, such as cardiac or heart tissue, in comparison to traditional injection methods without use of a substrate, resulting in reduced fibrosis and improved mechanical function. Sec J. P. Guyette et al., J. Biomed. Mater. Res. A, vol. 101A, no. 3, pp. 809-818, 2013. According to one aspect, the electrical impulse transmitting cells may be differentiated human stem cells. According to one aspect, the electrical impulse transmitting cells may be mesenchymal stromal cells. Moreover, exemplary fibrin threads are contemplated as the substrate, insofar as such threads have been used as a scaffold for tissue engineering with both rat ventricular cardiomyocytes in a cardiac patch and hiPSC-CMs in a contractile fiber. See M. O. Chrobak et al., ACS Biomater. Sci. Eng., vol. 3, no. 7, pp. 1394-143 July 2017 Exemplary bioelectric threads are crosslinked with a crosslinker to improve mechanical stability and handleability properties compared to uncrosslinked threads. According to one aspect, engineered cardiac tissue is coupled with bioelectric threads as described herein in vitro in a manner to create or re-engineer conduction paths in vivo.

FIG. 1 is a schematic of a bioelectric thread as described herein wherein a fibrin thread is either coated in (top) or embedded with (bottom) hiPSC-CMs, and action potentials are able to propagate from one end to the other. An exemplary bioelectric thread of biocompatible material such as fibrin is coated with one or more or a plurality of layers of cardiomyocytes including gap junctions to produce a bioelectric thread. An exemplary bioelectric thread is provided which includes a biocompatible material such as fibrin as a matrix material for electrical impulse transmitting cells, such as cardiomyocytes embedded therein including gap junctions to produce a bioelectric thread.

According to one embodiment, an exemplary core-shell thread is provided which includes a biocompatible material as described herein such as fibrin as a core and a shell of biocompatible material as described herein. A core-shell fiber comprises two separate fiber compartments; the outer compartment (denoted as “shell”), and the inner compartment (denoted as “core”), in which the latter is being completely enclosed by the former. Either the core or the shell or both the core and the shell may include electrical impulse transmitting cells, such as cardiomyocytes including gap junctions as shell to produce a bioelectric thread. Such core-shell bioelectric threads can be produced using co-extrusion methods known in the art. See M. Addullah et al., Polymers (Basel), 2019 Dec. 4; 11(12): 2008 hereby incorporated by reference in its entirety and the methods of co-extruding core-shell fibers.

An electrical signal is applied and action potentials are able to propagate through the bioelectric thread, such as from one end of the bioelectric thread to the other end of the bioelectric thread. According to one aspect, the bioelectric threads as described herein provide cardiomyocytes exhibiting an electrical syncytium prior to intramuscular implantation. According to one aspect, the present disclosure provides a method to deliver a bioelectric thread including cardiomyocytes, to the host myocardium and beyond the outer epicardial surface while limiting shearing of the cells from the substrate during installation of the bioelectric thread into muscle tissue.

### I. Biocompatible Support Material

According to one aspect, a substrate is provided which may include electrical impulse transmitting cells on the substrate or within the substrate. According to one aspect, the substrate includes or is otherwise formed from biologically acceptable or biologically compatible material, such as biologic material. Exemplary biologically acceptable or biologically compatible materials include biological materials such as fibrinogen, fibrin, collagen, gelatin, alginate, hyaluronic acid, chitosan, or decellularized extracellular matrix and the like or mixtures thereof. According to one aspect, biological support materials may be biodegradable. According to one aspect, the substrate is a fibrin substrate.

Other biologically acceptable or biologically compatible materials include biologically acceptable or biologically compatible synthetic polymers or copolymers as known in the art. Exemplary biologically acceptable or biologically compatible synthetic polymers or copolymers include polyethylene glycol, polylactic acid, polylactic-co-glycolic acid, polycaprolactone and the like or mixtures thereof. According to one aspect, such synthetic polymers or copolymers may be biodegradable.

According to one aspect, the substrate is in elongate form having a length between 1 cm and 12 cm, between 2 cm and 12 cm, between 3 cm and 12 cm, between 1 cm and 10 cm, between 1 cm and 9 cm, between 1 cm and 8 cm, between 1 cm and 7 cm, between 1 cm and 6 cm, between 2 cm and 12 cm, between 2 cm and 10 cm, between 2 cm and 9 cm, between 2 cm and 8 cm, between 2 cm and 7 cm, between 2 cm and 6 cm, between 3 cm and 10 cm, between 3 cm and 9 cm, between 3 cm and 8 cm, between 3 cm and 7 cm, between 3 cm and 6 cm, between 3 cm and 5 cm and having a diameter between 300 μm and 900 μm. The substrate may be referred to herein as a “thread,” e.g., a long thin strand or filament or fiber, insofar as the substrate resembles a thread or filament or fiber having a length exceeding a diameter. The thread may be cylindrical in form, though other forms are contemplated such as a flat strip or other elongated substrate form According to one aspect, two or more bioelectric threads may be combined to form a bioelectric thread bundle, whether by connecting bioelectric threads together or otherwise weaving bioelectric threads together and using such a bioelectric thread bundle as described herein.

According to one aspect, the biological support material may be crosslinked. According to one aspect, the biological support material may be crosslinked by a crosslinker. According to one aspect, crosslinking may be achieved by gelation and without a separate crosslinker. Exemplary crosslinkers include transglutaminase, thrombin, tannic acid, genipen, citric acid, or phytic acid and the like or mixtures thereof. According to one aspect, the crosslinked biological support material exhibits increased mechanical properties compared to the non-crosslinked biological support material, improving handleability of the bioelectric thread. According to one aspect, the substrate is a fibrin substrate, which may be referred to herein as a fibrin thread, is subject to a crosslinking reaction. The crosslinking reaction is used to crosslink the fibrin thread. According to one aspect, the crosslinked fibrin thread exhibits increased mechanical properties compared to the non-crosslinked fibrin thread, improving handleability of the thread. An exemplary crosslinker according to the present disclosure is transglutaminase.

An exemplary method of making fibrin threads is described in Hansen K J, Laflamme M A, Gaudette G R. Development of a Contractile Cardiac Fiber From Pluripotent Stem Cell Derived Cardiomyocytes. Front Cardiovasc Med. 2018 Jun. 11; 5:52. doi: 10.3389/fcvm.2018.00052. PMID: 29942806; PMCID: PMC6004416 hereby incorporated by reference in its entirety. Fibrin sutures are known in the art. See US20170182208. In general, methods of making fibrin threads contemplated herein include combining fibrinogen and thrombin and CaCl₂) and forming fibrin threads. Another method for making the bioelectric thread contemplated herein includes combining fibrinogen, gelatin, and cellular components to embed electrically conductive cells directly into the substrate to mechanically protect them from shear forces during delivery.

II. Electrical Impulse Transmitting Cells and Combining Electrical Impulse Transmitting Cells with Biocompatible Support Material

Electrical impulse transmitting cells according to the present disclosure include cardiomyocytes, cardiac conduction system cells, nodal cells, nerve cells, and/or muscle cells and the like or combinations thereof. Such electrical impulse transmitting cells may be differentiated from stem cells, such as pluripotent stem cells, such as induced pluripotent stem cells, such as human induced pluripotent stem cells. As an example, such stem cells may be differentiated into muscle cells, such as cardiomyocytes. The cardiomyocytes may exhibit electrical activity characteristic of native, healthy cardiomyocytes.

According to one aspect, one or more additional cell types may be utilized in combination with the electrical impulse transmitting cells. Such additional cell types include cardiac fibroblasts, epicardial cells, immune cells, endothelial cells, smooth muscle cells, vascular stromal cells, and/or pericytes and the like or combinations thereof.

Electrical impulse transmitting cells as described herein may be combined with, attached to or adhered to the biocompatible support material using methods known to those of skill in the art. According to one aspect a substrate in the form of a thread for example, is contacted with electrical impulse transmitting cells in a manner to form a layer of the electrical impulse transmitting cells on the surface of the substrate. Such an attachment or adherence method may be referred to herein as “seeding” as is known in the art. The methods described herein and known in the art result in cells being attached or adhered to the surface of the support. According to one aspect, one or more or a plurality of layers of cells are contemplated. According to one aspect, the substrate is seeded with cells in a density of ranging between 5×10\hat{ }5 cells to 4×10\hat{ }6 cells per centimeter of fibrin thread length to establish an electrically conductive thread. In general, fibrin threads prepared as generally described above are stretched and dried followed by contacting the threads with 75%-95% hiPSC-CMs and 5%-25% hCFs in a manner to “seed” or adhere the cells to the fibrin thread. The seeded threads are then subject to culture conditions and electrical stimulation.

According to another aspect, cells are embedded directly in the substrate, referred to herein as “embedding” as is known in the art. The substrate is seeded with 75-95% hiPSC-CMs and 5-25% hCFs in a density between 15 and 50×10\hat{ }6 total cells per milliliter of total material. The embedded threads are then subject to culture conditions and electrical stimulation.

According to one aspect, the electrical impulse transmitting cells, such as cardiomyocytes, may be differentiated from stem cells, human stem cells, induced pluripotent stem cells or human induced pluripotent stem cells. Exemplary cardiomyocytes include ventricular cardiomyocytes, atrial cardiomyocytes, sinoatrial node cardiomyocytes, atrioventricular node cardiomyocytes, bundle of His cardiomyocytes, bundle branch cardiomyocytes, and the like or combinations thereof.

According to one aspect, a mixture of substrate material and electrical impulse transmitting cells, such as cardiomyocytes, is made and a thread or fiber is formed from the mixture.

According to one aspect, a core-shell structure of a core of biomaterial and a shell of electrical impulse transmitting cells, such as cardiomyocytes, is made using a core-shell co-extrusion process, as is known in the art.

### III. Electrical Activity of the Bioelectric Threads

According to one aspect, the bioelectric threads as described herein exhibit electrical activity. According to one aspect, the bioelectric threads as described herein exhibit electrical activity and can be used to bridge electrical activity across two locations of tissue, such as cardiac tissue or between two separate tissues. Matching the cell type, in the presented case cardiomyocytes, to the organ intended for delivery, in the presented case the heart, enables electrical signals to be propagated at comparable velocities as the host organ, minimizing risk for incompatibilities and development of aberrant electrical rhythm. Exemplary cell types include atrial cardiomyocytes and ventricular cardiomyocytes, for chamber specific application in the heart and neurons for brain and muscle applications. According to one aspect, the elongated form of the bioelectric thread, can be stretched at varying levels of strain to increase alignment and organization of cells attached on the substrate to further increase conduction velocities.

According to one aspect, bioelectric threads as described herein are cultured in either standard media or maturation media to become electromechanically active. As shown in FIG. 2, applying point electrical stimulation at one end of the bioelectric thread uniformly propagates electrical signal to the opposite end, up to a frequency of 3 Hz or 180 beats per minute, well beyond that necessary for the healthy range of human heart rates during intense exercise. As shown in FIG. 3 and FIG. 4, bioelectric threads including a fibrin thread with cardiomyocytes seeded thereon and cultured produce a frequency-dependent electrophysiological response, with threads cultured in maturation media exhibiting significantly faster conduction velocities compared to threads cultured in standard media.

IV. Delivery of the Substrate Seeded with Cells

According to one aspect, an injection-based delivery system is used to insert bioelectric thread into muscle tissue. According to one aspect, the bioelectric thread is contained within a tube with a portion of the fibrin thread exposed from the tube. The portion of the thread exposed from the tube is referred to as an anchor portion, insofar as the anchor portion serves to anchor the bioelectric thread within the tissue. The anchor portion may be in the shape of a curve or hairpin. The anchor portion of the fibrin thread exposed from the tube may include cells or it may not include cells. The tube is inserted into a needle or cannula or other delivery device as is known in the art for injection within tissue. The needle is inserted into muscle tissue. The portion of the fibrin thread exposed from the tube, i.e. the anchor portion, serves to provide a frictional anchor or resistance to keep the biological thread from being removed from the tissue upon needle extraction. Delivery may or may not include ejection from the needle via application of a plunger or other mechanism providing force as is known in the art. Delivery may or may not include ejection from the needle via application of a plunger or other mechanism providing force to a delivery liquid as is known in the art. The portion of the fibrin thread exposed from the needle acts as a hook to help secure the biological thread within the tissue. According to one aspect, the needle and tube may be withdrawn simultaneously thereby leaving the biological thread within the tissue. According to one aspect, the needle may be withdrawn leaving the tube and the bioelectric thread within the tissue. The tube may then be withdrawn thereby leaving the biological thread within the tissue. According to one aspect, a lubricant may be within the tube to facilitate withdrawal or ejection of the bioelectric thread from within the tube while minimizing removal of seeded cells from the bioelectric thread during the withdrawal or extraction due to friction between the tube and the bioelectric thread. Lubricants within the scope of the present invention include biologically compatible lubricants known to those of skill in the art. Exemplary lubricants contemplated by the present disclosure include water-based lubricants such as Surgilube™ and oil-based lubricants such as Muri-lube™. According to one aspect, the interior of the tube may have a friction resistant-surface or a friction-resistant surface applied thereto, thereby avoiding the need for a lubricant.

According to one aspect, the tube has dimensions sufficient to have the bioelectric thread occupied therein. Accordingly, the tube may have a length between 1 cm and 12 cm, between 2 cm and 12 cm, between 3 cm and 12 cm, between 1 cm and 10 cm, between 1 cm and 9 cm, between 1 cm and 8 cm, between 1 cm and 7 cm, between 1 cm and 6 cm, between 2 cm and 12 cm, between 2 cm and 10 cm, between 2 cm and 9 cm, between 2 cm and 8 cm, between 2 cm and 7 cm, between 2 cm and 6 cm, between 3 cm and 10 cm, between 3 cm and 9 cm, between 3 cm and 8 cm, between 3 cm and 7 cm, between 3 cm and 6 cm, between 3 cm and 5 cm. According to one aspect, the tube may have a diameter between 300 μm and 900 μm. In any event, the tube has sufficient length and diameter to accommodate the bioelectric thread therein, and with a portion exterior to the tube as described herein.

The needle has dimensions sufficient to have the bioelectric thread occupied therein. Accordingly, the needle may have a length between 1 cm and 12 cm, between 2 cm and 12 cm, between 3 cm and 12 cm, between 1 cm and 10 cm, between 1 cm and 9 cm, between 1 cm and 8 cm, between 1 cm and 7 cm, between 1 cm and 6 cm, between 2 cm and 12 cm, between 2 cm and 10 cm, between 2 cm and 9 cm, between 2 cm and 8 cm, between 2 cm and 7 cm, between 2 cm and 6 cm, between 3 cm and 10 cm, between 3 cm and 9 cm, between 3 cm and 8 cm, between 3 cm and 7 cm, between 3 cm and 6 cm, between 3 cm and 5 cm. According to one aspect, the needle may have a diameter between 300 μm and 900 μm. In any event, the needle has sufficient length and diameter to accommodate the bioelectric thread or the tube therein. Exemplary needle gauges include between 12 to 20 gauge.

According to one aspect, the needle may include two longitudinal, interlocking, and separable portions between which the bioelectric thread is positioned. The purpose of the two longitudinal, interlocking, and separable portions of the needle is to facilitate positioning of the bioelectric thread within the needle, insofar as one half of the needle is exposed to allow for easy placement of the bioelectric thread within the one half of the needle and the other half of the needle is placed on top of and secured to form the complete needle structure. According to one aspect, the longitudinal, interlocking, and separable portions may be separated, or “opened” and the bioelectric thread may be position on one of the longitudinal portions, and the two longitudinal portions may be combined together or “closed” and interlocked. According to one aspect, the bioelectric thread may be within a tube as described herein before being placed within the needle including the two longitudinal, interlocking, and separable portions. The needle is inserted into muscle tissue and the needle is withdrawn leaving the bioelectric thread within the tissue. The portion of the bioelectric thread exposed from the tube, i.e. the anchor portion, which may be in the shape of a curve or hairpin serves to provide resistance to the bioelectric thread from being removed from the tissue upon needle extraction. The portion of the fibrin thread exposed from the tube as a hook helps secure the bioelectric thread within the tissue. According to one aspect, the needle and tube, if present, may be withdrawn simultaneously thereby leaving the bioelectric thread within the tissue. According to one aspect, the needle may be withdrawn leaving the tube and the bioelectric thread within the tissue. The tube may then be withdrawn thereby leaving the bioelectric thread within the tissue. With reference to FIG. 5, an embodiment of a needle having two longitudinal, interlocking, and separable portions between which the bioelectric thread is positioned is shown generally at 10. Two half portions 20 and 30 are shown separated and with the bottom half having a bioelectric thread 60 placed therein. The interlocking mechanism is depicted at groove 40 and corresponding hook 50. Groove 40 on top portion 20 fittingly engages with hook 50 of bottom portion 30 as shown in the top images left to right of FIG. 5. The right most image shows the grove 40 interlocked with the corresponding hook 50. The top and bottom separable portions of the needle include two interlocking mechanisms on opposite sides of the separable needle portions as shown in FIG. 5. Also shown in FIG. 5 is threaded section 70 which is used for threadingly engaging an apparatus, such as a syringe or needle hub.

### Example I

hiPSC Maintenance

WTC-11 GCaMP6f-expressing human induced pluripotent stem cells (hiPSCs; Bruce Conklin, Gladstone Institute, UCSF) were seeded in 10 cm² dishes coated with 5 μg/mL vitronectin (VTN-N, Thermo Fisher Scientific, Waltham, MA, USA). Cells were maintained in Essential 8 medium (E8) in a cell culture incubator (5% CO2, 37° C.) for 4 to 5 days. Once 80% confluency was reached, cells were harvested using versene (0.5 M EDTA (Fisher) and 1.1 mM D-glucose (MilliporeSigma) in Dulbecco's Phosphate-Buffered Saline (DPBS) without calcium and magnesium (Gibco)) and plated at a 1:10 split ratio onto new VTN-N coated plates.

### Example II

### Cardiomyocyte Differentiation, Expansion, and Lactate Selection

Before differentiation, hiPSCs were singularized and replated onto Matrigel (Corning) or Geltrex-coated (Gibco) 24-well plates in E8 media with 5 μM Y-27632 (ROCK inhibitor, RI; Tocris, Bristol, UK). The following day (day 0), cells were treated with 4.5 μM CHIR 99021 (Chiron; Tocris, Bristol, UK) in cardiac differentiation medium with 3 components (CDM3), RPMI 1640 (Gibco) supplemented with 213 μg/mL L-ascorbic acid and 500 g/mL human serum albumin, for 24±1 hours. Plates were washed with DPBS and fed with fresh CDM3 on day 1. On day 3 of differentiation, 72 hours after the initial treatment with CHIR, cells were treated with 5 μM IWP2 (Tocris) in half fresh and half spent CDM3 media. On day 5 of differentiation, IWP2 was removed after 48±1 hours, and the culture media was replaced with fresh CDM3 media every other day until day 9 when beating began.

HiPSC-derived cardiomyocytes (hiPSC-CMs) were either harvested for cryogenic storage and expansion on day 11 or replated on day 14. One hour prior to harvest, 5 μM RI was added to each well. Dishes were washed with DPBS and incubated with TrypLE™ Select Enzyme (TrypLE10x, Gibco) for 20 minutes. Cells were then triturated and added to an equal volume of RPMI 1640 media with B27 supplement (RPMI/B27; Gibco), 10% fetal bovine serum (FBS; Gibco), and 100 kU/mL DNAse I (DNase; Thermo Fisher Scientific, Waltham, MA, USA). After centrifugation for 5 minutes at 300 g, DNAse was added directly to the cell pellet before adding additional RPMI/B27 media and FBS. Cells were counted with a hemocytometer and centrifuged again.

HiPSC-CMs for cryogenic storage and subsequent expansion were resuspended at 5 M/mL in CryoStor® CS10 (Stem Cell, Vancouver, Canada) and distributed into cryogenic vials that were then placed in room temperature CoolCell Freezing Containers (Corning) and frozen at −80° C. overnight. The next day, cell vials were moved to storage in liquid nitrogen. Eventually, vials were thawed in a 37° C. water bath before adding the cells to a collection tube of RPMI/B27 media and centrifuging for 5 minutes at 300 g. The supernatant was aspirated and cells were resuspended in RPMI/B27. For expansion of terminally differentiated hiPSC-CMs, cells were seeded onto Geltrex-coated 15 cm plates at low density, approximately 300,000 hiPSC-CMs/cm², in RPMI/B27 media with 5 μM RI. A low concentration of Wnt activation (2 μM CHIR) was introduced into RPMI/B27 feeding media every other day between days 13 and 19 for 4 to 6 days until 90% confluency was achieved.

Alternatively, cells may not undergo expansion and instead were replated onto Matrigel or Geltrex-coated 24-well plates in RPMI/B27 media with 10% FBS and 5 μM RI. Media was changed to fresh RPMI/B27 the next day, and cells were fed every other day. On day 19 or 20, metabolic selection of cardiomyocytes was initiated for both expanded and non-expanded cells. Feeding medium was switched to Dulbecco's Modified Eagle Medium (−glucose) (DMEM (−)glucose; ThermoFisher Scientific) supplemented with 4 μM sodium lactate (MilliporeSigma) for 4 days. Post selection, all cells were fed with RPMI/B27 in a recovery period. Lastly, expanded cells received C59 4 μM treatment on day 25. Post lactate purification, cardiomyocytes were fed every 3 days and cultured up to day 40. See FIG. 6A depicting a non-expanded differentiation process. See FIG. 6B depicting an expanded differentiation process with a free/thaw step.

### Example III

### Human Cardiac Fibroblast Maintenance

Human primary cardiac fibroblasts (hCFs; MilliporeSigma) were cultured on 10 cm² dishes in Dulbecco's Modified Eagle Medium/Nutrient Mixture F-12 (DMEM/F-12 media; Gibco) with 10% fetal bovine serum (FBS; Gibco), 100 μg/mL Penicillin-Streptomycin (pen-strep; MilliporeSigma, Cleveland, OH, USA), and 4 ng/ml basic fibroblast growth factor (bFGF; Stemgent). Cells were passaged at 80% confluency every 4 to 5 days using 0.05% trypsin (Gibco) in versene and resuspended in 10% v/v dimethyl sulfoxide (DMSO; Fisher Scientific, Waltham, MA, USA) for cryopreservation. hCFs were immediately frozen at −80° C. overnight and moved to a cryotank for long term storage the next day. All experiments were performed using hCFs from passages 4 to 5.

### Example IV

### Flow Cytometry

Cells from each differentiation were analyzed using flow cytometry to determine the percent cardiomyocyte population, a metric of differentiation purity. Single-cell suspension samples were fixed using 4% (v/v) paraformaldehyde in the dark at room temperature for 10 minutes. Cells were then stained for alpha smooth muscle actin (αSMA, 1:200; Abcam) and cardiac troponin T (cTnT; 1:500; Thermo Fisher Scientific). Samples were processed using a BD FACSAria Flow Cytometer and analyzed using either FlowJo or Flowing Software.

### Example V

### Fibrin Thread Extrusion and Crosslinking

In order to manipulate the mechanical properties of fibrin, transglutaminase was used to crosslink the threads. Lyophilized powder thrombin from bovine plasma (Sigma-Aldrich; Catalog #T4648-1KU) was solubilized to 40 U/mL in PBS. 40 mM calcium chloride (CaCl2; Acros Organics) in ultrapure Milli-Q filtered water (Milli-Q; Millipore Sigma) was added to the thrombin to make a working concentration of 8 U/mL. Fibrinogen from bovine plasma (Sigma-Aldrich; Catalog #F8630-5G) was solubilized to 50 mg/mL by layering it above warm (37° C.) DPBS and dissolving for 6 hours with no agitation until it became a hazy solution. The thrombin and fibrinogen solutions were drawn into 1 mL insulin syringes and extruded through a double syringe extrusion tip on a Harvard Apparatus Standard Infusion Only 11 Elite Syringe Pump. Upon introduction to each other, the thrombin triggered a cascade of reactions converting fibrinogen to fibrin. The infusion rate was set to 0.23 mL/min, and the combined solution was ejected through a 30 gauge hypodermic needle and 38 cm of medical grade polyethylene tubing (I.D.×O.D.: 0.38 mm×1.09 mm; Scientific Commodities Inc.) into a 10 mM 4-(2-Hydroxyethyl) piperazine-1-ethanesulfonic acid, N-(2-Hydroxyethyl) piperazine-N′-(2-ethanesulfonic acid) (HEPES; Sigma-Aldrich Catalog #H3375) buffer in 40 mM CaCl2. The fibrin threads were left in the HEPES bath for 1 hour in order to create dense fibrillar fibrin networks before being stretched and dried overnight.

A 200 mg/mL stock solution was prepared by dissolving and sterile filtering transglutaminase (TG; Moo Gloo) in DPBS. This solution was then serially diluted to 200 mg/mL, 20 mg/mL, 2 mg/mL, 0.2 mg/mL, 0.02 mg/mL, and 0.002 mg/mL, and threads were submerged for 60 minutes to crosslink the fibrin. Threads were then dried overnight once more.

According to one embodiment, a method is provided for extrusion-based fabrication of bioelectric threads. Differentiated, lactate-purified cardiomyocytes are harvested and counted. Human cardiac fibroblasts are harvested or thawed from previously frozen human cardiac fibroblasts. A material mixture is created from 5%-20% gelatin, 10 mg/ml to 40 mg/ml fibrinogen, 4%-7% v/v 10×RPMI, and 0.5%-2.0% v/v HEPES. A cell suspension is created of 30M to 150M cells per mL in RPMI/B27 including 75%-95% human induced pluripotent stem cell derived cardiomyocytes and 5%-25% human cardiac fibroblasts. Two parts of the material mixture is combined with one part of the cell suspension (2:3 material mixture, 1:3 cell suspension). The cell-material-crosslinker composite mixture may be combined within the body of a syringe and extruded through a needle into a polydimethylsiloxane (PDMS) mold or onto a cooled glass plate. Needle-based extrusion may be conducted manually or automatically using a bioprinter. The mold is then covered with RPMI/B27 or maturation media plus 10 uM Y-27632 ROCK inhibitor. A supplementary crosslinker, such as 2 U/mL to 250 U/mL of thrombin, may be added to the composite mixture immediately prior to extrusion or included in the media to cover the tissue post-extrusion. Compaction and remodeling occur over the next 5 to 7 days. Media is refreshed one day after fabrication and then every other day. After 5 to 7 days allowing for compaction, the thread can then be removed from the mold and electromechanically conditioned. Electromechanical conditioning can include static stretch, pulsatile stretch or no stretch culture with either field or point stimulation electrical conditioning, as is known in the art. FIGS. 7A and 7B depict bioelectric threads produced by the above method, with the thread statically stretched to up to 60% strain by lengthening and pinning threads with minutien pins onto a PDMS base and field stimulated. Electromechanical conditioning may also be performed using a culture bioreactor. FIG. 8 illustrates a uniaxial mechanical and electrical conditioning bioreactor that can be used in the methods described herein. FIG. 9 is a scanning electron microscope image demonstrating successful remodeling and alignment of cells along the electromechanical conditioning axis. FIG. 10 are immunohistochemical images of cellular alignment of stretched and nonstretched bioelectric threads, depicting significant improvement in cellular alignment with stretch. Blue represents staining with Hoechst while red represents staining with A-actinin, a cytoskeletal marker. FIG. 11 and FIG. 12 demonstrate that stretched bioelectric threads significantly increased conduction velocity (CV) and maximum capture rate (MCR), likely attributed to improved cellular alignment and polarization of gap junction.

### Example VI

### Tensile Testing of Threads

A single column Instron 5940 with a 500 N load cell was used to measure the tensile properties of the acellular fibrin threads with a displacement rate of 10 mm/min. Initial testing was performed on fibrin threads after 15 minutes in DPBS. In the second iteration of testing, the samples were added to thread media consisting of 88% RPMI/B27, 10% FBS, and 2% penicillin streptomycin amphotericin B (pen-strep-amphotericin B; 10,000 Units/ml, 10,000 μg/ml, 25 μg/ml; Thermo Fisher Scientific) and placed in a cell culture incubator. Tensile testing to failure for these threads was conducted after 1 hour, 12 hours, 1 day, 2 days, 5 days, 8 days, and 12 days of incubation. Thread diameters were measured with image analysis, and gauge length, displacement, and load were recorded to generate stress-strain curves. Young's modulus was then calculated using the linear stress-strain relationship, and ultimate tensile strength was noted as the maximum stress achieved before failure.

Mechanical differences between fibrin threads and synthetic suture material was investigated. When compared to suture material (7-0 polypropylene) often used on the heart, fibrin threads exhibited lower mechanical stiffness and strength, though fibrin threads may still be useful in some applications. FIG. 13A depicts comparative data of the diameter of dry fibrin threads, wet fibrin threads and 7-0 polypropylene. FIG. 13B depicts comparative data of Young's modulus (MPa) for dry fibrin threads, wet fibrin threads and 7-0 polypropylene. FIG. 13C depicts comparative data for ultimate tensile strength for dry fibrin threads, wet fibrin threads and 7-0 polypropylene. The wet condition was particularly of interest since threads are cultured in cell media prior to handling and in vivo delivery.

Further tensile testing of the fibrin threads illustrated significant differences in mechanical properties after crosslinking in 200, 20, 2, 0.2, 0.02, and 0.002 mg/mL of transglutaminase. FIG. 14A depicts a tensile strength testing setup. Threads were placed between two pieces of double-sided tape and loaded into a single column Instron 5940 with a 500 N load cell. FIG. 14B is a graph of representative force (mN) vs. displacement (mm). FIG. 14C depicts data of Young's modulus calculated using the initial linear stress-strain curve derived from the force vs. displacement data and measured gauge lengths. FIG. 14D depicts data of ultimate tensile strength defined as the maximum stress on the specimen before failure (n=3 for all conditions).

The maximum stress achieved before fracture indicated statistically insignificant changes in ultimate tensile strength after crosslinking; however, a prominent trend was observed. Difficulty handling and manipulating the acellular threads while loading into the TPU tubes were associated with the 200 mg/mL, 0.02 mg/mL, and 0.002 mg/mL conditions. The 200 mg/mL condition was particularly brittle and could not be manipulated with forceps while the 0.02 mg/mL and 0.002 mg/mL conditions had no noticeable differences from the control.

Tensile testing of the control, 2 mg/mL, and 20 mg/mL biomaterial only fibrin threads was continued for 12 days. Decreases in Young's modulus were prominent after 12 hours, suggesting the fibrin thread was not fully saturated at the initial hour. Meanwhile, the ultimate tensile strength was consistent, suggesting that there would not be a significant compromise to the material strength of fibrin from culture conditions. FIG. 15A is a graph of Young's Modulus (MPa) from Day 0 to Day 12. FIG. 15B is a graph of ultimate tensile strength (mPa) from Day 0 to Day 12.

Image analysis of all threads from day 12 revealed significant differences in diameter, suggesting that cardiomyocyte compaction is not only dependent on cell purity, but also mechanical properties of the fibrin thread. The bioelectric threads from the 2 mg/mL group showed the smallest diameter in all four batches of bioelectric threads. It is hypothesized that the stiffnesses of the fibrin thread impacted the ability of CMs to migrate and align, thus influencing compaction and diameter. FIG. 16 depicts data of image analysis using Image J of the diameters of bioelectric threads. Dashed lines represent the average diameter of hydrated acellular threads on day 12 (255 μm) and the inner diameter of the tube used in the delivery device (550 μm). Significance is defined as *p<0.5; ***p<0.001, ****p<0.0001.

### Example VII

### Delivery Device Preparation

Bioelectric threads are loaded into an 18-gauge needle using microcatheter thermoplastic polyurethane tubing (TPU, I.D.×O.D.: 0.55 mm×0.75 mm; Raumedic) into which the bioelectric thread is positioned with a portion of the bioelectric thread exposed and exterior to the tubing. The loaded needle is then inserted into the myocardium with the portion of the bioelectric thread exterior to the tubing being an exposed acellular, fibrin-only tail in order to be held in place by friction upon removal of the needle. The thread is positioned as the needle is withdrawn simultaneously with the tubing, leaving the bioelectric thread in the host tissue and enabling the hiPSC-CMs to electrically couple with the host cardiomyocytes. According to one aspect, the thread is positioned as the needle is withdrawn ejecting the tubing with the bioelectric thread within. Then, the tubing is withdrawn with the bioelectric thread remaining within the tissue and enabling the hiPSC-CMs to electrically couple with the host cardiomyocytes.

In order to load the threads into the TPU tubes, a rig was developed using Fusion 360 and 3D printed with polylactic acid filament in order to cut 30 mm length slits down 35 mm strips of the tubing, referred to herein as a tube cutting rig design. FIG. 17A depicts in schematic two 35 mm blocks with a channel cut down the middle of each block to accommodate a polyurethane tube. The two blocks are the placed together to enclose a polyurethane tube. The two blocks placed together are then placed into a holder depicted in FIG. 17B and a blade is used to cut the tubing.

### Example VIII

### Injection Testing in Surrogate Muscle Tissue

The injection-based delivery device described herein was tested using biomaterial-only fibrin threads injected into chicken breast acting as surrogate fibrous muscle tissue. The uncrosslinked control condition was tested in addition to crosslinked fibrin threads crosslinked with 0.2 mg/mL, 2 mg/mL, and 20 mg/mL transglutaminase crosslinking conditions. Threads were tested with positioning transverse and parallel to muscle fiber alignment.

An exposed tail end for positioning of the thread was advantageous during injection testing into the chicken muscle tissue. Successful injection into chicken muscle tissue was achieved using the 2 mg/mL and 20 mg/mL transglutaminase treated crosslinked threads. The control (noncrosslinked) and 0.02 mg/mL transglutaminase conditions did not produce a thread stiff enough to facilitate positioning into the tubing and accordingly could not be injected into the chicken muscle tissue.

According to one aspect, the tubing and needle are withdrawn together leaving the thread within the muscle tissue. According to one aspect, the needle is withdrawn while leaving the tubing in place, followed by withdrawing the tubing leaving the thread within the muscle tissue. As shown in schematic in FIG. 18A, a bioelectric thread is placed into sliced tubing. As shown in schematic in FIG. 18B, the tubing is loaded into an 18-gauge needle. FIG. 18C is an image of the delivery device (thread within sliced tubing within a needle) with the acellular thread exposed and having a curved tail portion outside of the sliced tubing. As shown in FIG. 18D in schematic, the needle with the sliced tubing therein and with the bioelectric thread within the sliced tubing is injected into the host tissue. The needle is removed, ejecting the tubing which is then removed separately and leaving behind the bioelectric thread within the host tissue. An exposed biomaterial-only tail portion (e.g, without cell seeding) is used to hold the bioelectric thread in place within the host tissue. It is contemplated that the tail portion can be seeded as desired.

### Example IX

### Bioelectric Thread Fabrication and Maintenance

Fibrin threads were coated in Matrigel and stored in a humidity chamber at 4° C. for 24 to 48 hours prior to seeding cells. Threads were removed from the humidity chamber and left at room temperature 1 hour before seeding. hiPSC-CMs were harvested and mixed with 5% hCFs. Cells were suspended with thread media supplemented with 0.05 mg/mL of aprotinin, a protease inhibitor used to slow fibrinolysis, to create a solution of 10 M cells/mL. A singular fibrin thread was placed in each well of a polydimethylsiloxane (PDMS; 184 Silicone Elastomer Clear; Dow SYLGARD) mold. FIG. 19A depicts illustrations and dimensions of a cell-seeding mold. FIG. 19B depicts a rendering of a negative PDMS mold developed in Onshape. FIG. 19C depicts a PDMS cell-seeding mold made from a negative PDMS mold. 200 μL of the cell solution was added to the cell-seeding mold, ensuring the thread was not floating and removing any bubbles. Bioelectric threads were then fed in the cell-seeding mold for 5 to 6 days before being transferred to a 6-well plate. Electrical stimulation at 1 Hz was applied from day 5 to at least 12, during which tissues were fed with new thread media and aprotinin every other day.

FIG. 20 depicts in schematic a method for bioelectric thread fabrication. Thrombin and fibrinogen form fibrin threads through a coagulation cascade before they are crosslinked in transglutaminase and coated in Matrigel. Threads and cells are added to the PDMS mold and cultured 5 days before receiving electrical stimulation.

Bioelectric threads were fabricated four times, and hiPSC-CMs differentiations were held consistent between each batch. Flow cytometry results indicated small differences in cardiomyocyte purity (% cTnT+) between cardiac directed differentiations (CDDs) (See FIG. 21 which provides representative flow cytometry data). Batch #1 of the threads consisted of 4 CDDs with an average purity of 81.5%. Next, batch #2 and #3 were made from expanded cardiomyocytes, with purities of 71.91% and 89.41% respectively. Lastly, batch #4 used 2 CDDs averaging 75.84%, indicating that all bioelectric threads were seeded with cardiomyocyte populations with purities greater than 70%. See Table 1 below.

TABLE 1

| Batch Number | Purity | Expansion |
|---|---|---|
| 1 | 81.5 | No |
| 2 | 71.91 | Yes |
| 3 | 89.41 | Yes |
| 4 | 75.84 | No |

However, batch 2 resulted in the most non-uniform seeding of hiPSC-CMs around the fibrin threads, likely due to compaction of non-cardiomyocytes. A lesser difference was observed in batch 4, suggesting that purities greater than approximately 80% would be optimal for the most successful fabrication.

As early as 2 days after introducing hiPSC-CMs to the fibrin threads, beating was observed as tissues compacted. However, minimal compaction was observed in some threads, resulting in limited attachment (see FIG. 22A showing representative images of threads and cell compaction on threads in seeding molds. Thus, these threads were unable to be transferred from the seeding molds to 6-well plates from electrical stimulation, demonstrating a failure mode. Between all four batches of bioelectric threads, the 2 mg/mL demonstrated the highest success rate (see FIG. 22B depicting comparative success rate data of the control, 2 mg/ml crosslinked thread and the 20 mg/ml crosslinked thread).

### Example X

### Cell Viability Assay

On day 12, bioelectric threads were placed in polyurethane tubing with a tail hanging out of one end. The tubing was pulled away while the tail was held with forceps, mimicking the frictional force applied by surrounding tissue in the proposed delivery system. The tubing and threads were immediately returned to thread media with aprotinin and stained with a two-color nuclear staining assay for cell viability (ReadyProbes™ Cell Viability Imaging Kit, Blue/Red; Invitrogen; ThermoFisher Scientific). Two drops of the staining solution were added per mL of media, and samples were incubated for 15 minutes. Images of each thread and tube were taken using a Nikon Eclipse Ti widefield microscope.

### Example XI

### Testing of Delivery Mechanism

Threads were placed in TPU tubes and removed using forceps as previously described for the cell viability assay. Immediately after, the tubes were incubated in TrypLE for 15 minutes. The tissue fragments were then triturated and removed from the tubing. The cell suspension was added to an equal volume of CDM3, and tubing was rinsed with CDM3. The collected cells were centrifuged for 5 minutes at 300 g. The pellet was resuspended in CDM3 and triturated into a single cell suspension, and cells were counted with a hemocytometer. The same was performed after coating the inside of TPU tubes with surgical lubricant (Surgilube). Lubricants were investigated due to their common use in reducing friction during the insertion of catheters. This particular sterile surgical lubricant was chosen in comparison to other products and lubrication methods due to the ingredients listed in its safety data sheet and its commercial availability. While other products like Surgical Lubricating Jelly (Medline) contain large amounts of 1,2,3-Propanetriol, Surgilube consists of water, hypromellose, propylene glycol and chlorhexidine gluconate, which makes the product bacteriostatic.

Ejection of the thread from the delivery device was simulated by removing the thread from the tubing using forceps. In order to load the thread into the tubing, a single edge blade razor was used in conjunction with the 3D-printed tube cutting rig to slice the TPU tube lengthwise. A bioelectric thread was then carefully positioned inside, and the tube was then placed into an 18G needle to prevent shear upon injection. FIG. 23 depicts in schematic loading of the bioelectric thread into the sliced tubing with a portion of the thread without cell seeding exposed from the sliced tubing. The tubing having two separable portions with a bioelectric thread position between is depicted in FIG. 18A. According to this aspect, an embodiment is provided including a tube which has a portion separated into two half portions with a remaining portion of the tube intact as an intact cylindrical portion. It is to be understood that the two half portions need not be exactly one half, just so that one longitudinal portion of the tube may be separated from the remaining longitudinal portion of the tube. The term “half” is used as a matter of convenience. The two half portions are separated and the bioelectric thread is placed into one of the half portions and the two half portions are placed together to reform the tube. The tube is then placed into the needle with the portion of the tube in two half portions proximate the end portion of the needle.

FIG. 24A-D are directed to cell counts on threads and tubes after ejection testing. FIG. 24A-B are directed to quantifying the number of cells remaining in each tube and on threads after ejection and without using a lubricant using a dissociation agent and counting cells with a hemocytometer. The absence of cells remaining on the thread was confirmed with immunohistochemistry (Control n=2, 2 mg/mL n=4, 20 mg/mL n=3). FIG. 24C-D are directed cell counts on threads and tubes after ejection testing but using a lubricant. (Control n=3, 2 mg/mL n=1, 20 mg/mL n=2).

Cell count conducted on the tissues remaining in the tubes indicated an average of 299,278±161,983 cells, out of the initial 2×10⁶ cells seeded, would be successfully delivered if the shearing factor was removed (see FIG. 24A-B). The use of surgical lubricant decreased the detachment of cells from the fibrin (see FIG. 24C-D). Accordingly, one aspect of the present disclosure is the use of a lubricant to reduce friction or shear within the tube so as to inhibit removal of cells from the bioelectric thread upon removal of the bioelectric thread from the tube.

### Example XII

### Immunofluorescent Imaging for Bioelectric Thread Characterization

Samples were fixed in 4% paraformaldehyde in PBS for 15 minutes, rinsed in PBS, and stored in 30% (v/v) sucrose overnight. The following day, threads were embedded in frozen blocks of Optimal Cutting Temperature Compound (OCT; Tissue Tek; Sakura) and sectioned into 10 μm thick sections. Slides were stored at −20° C. until ready for staining.

Frozen sections were rinsed with PBS 3 times prior to performing a proteinase K digest antigen retrieval for 10 minutes. Immediately after, slides were rinsed in PBS and incubated in 1.5% normal goat serum in PBS for 1 hour to block non-specific binding. Samples were incubated in primary antibodies against cardiac troponin T (cTnT; 1:150; Thermo Fisher Scientific) or alpha actinin (α-actinin; 1:200; Abcam) and alpha smooth muscle actin (αSMA, 1:100; Abcam) or Connexin 43 (Cx43; 1:200; Sigma) overnight at 4° C. before being washed in PBS 3 times. Sections were then incubated for 1 hour at room temperature with secondary antibodies conjugated to Alexa Fluor 594 (AF594; 1:250; LifeTech) or Alexa Fluor 488 (AF488; 1:250; LifeTech). All samples were counterstained with Hoechst 33342 (1:1000; Invitrogen) and rinsed in PBS 3 times before being mounted with coverslips and ProLong Gold Antifade Mountant (Invitrogen). Slides were imaged using an Olympus VS200 Slide Scanner or Olympus FV3000 Confocal Microscope and processed with ImageJ.

Fluorescence imaging of stains for cTnT confirmed the presence of cardiomyocytes, and alpha-smooth muscle actin verified the existence of non-cardiomyocyte cells, including cardiac fibroblasts. Alpha-actinin and connexin 43 staining was performed to highlight the presence of organized sarcomeres and the initiation of gap junction formation, respectively (see FIG. 25A-D). Sarcomeres appeared to be most organized in the 2 mg/mL condition, suggesting cardiomyocytes were able to migrate and align best on these fibrin threads. Minimal initial formation of gap junctions was seen with connexin 43, but increased expression is expected when cultured for longer periods with electrical stimulation.

FIG. 25A depicts immunohistological staining of a control thread. FIG. 25B depicts immunohistological staining of a 2 mg/mL crosslinked thread. FIG. 25C depicts immunohistological staining of a 20 mg/mL crosslinked thread. The threads were stained against alpha-actinin and connexin 43 and counterstained for Hoechst. FIG. 25D depicts immunohistological staining of a control thread stained against cardiac troponin T and alpha-smooth muscle actin with Hoechst counterstain.

### Example XIII

### Optical Mapping

To assess if crosslinking threads with transglutaminase (TG) altered the electrical behavior of adhered hiPSC-CMs, optical mapping was performed. Optical mapping is a high-resolution fluorescence-based imaging technique often used in the field of cardiac electrophysiology to capture voltage traces of action potentials (APs) and their propagation across tissue. It is contemplated that the bioelectric threads described herein keep up with human heart rates and relay electrical signal with speeds comparable to the native human heart. Optical mapping was used to validate that an electrical syncytium sufficient enough to facilitate action potential propagations could be achieved in crosslinked threads without prolonging action potential duration (APD) or decreasing conduction velocity (CV).

Control threads and threads crosslinked with either 2 or 20 mg/mL TG were removed from culture media, rinsed with PBS, and loaded with a voltage-sensitive dye (5 M di-4-ANEPPS; Fisher Scientific) for 1 minute. Residual dyes were washed out completely with PBS before threads were placed onto a temperature regulated imaging stage (35-37° C., WPI) in mini petri-dishes in a bath of modified Tyrode's solution containing (in mM) 140 NaCl, 5.1 KCl, 1.0 MgCl 2, 1.0 CaCl 2, 0.33 NaH 2 PO 4, 5 HEPES, and 7.5 glucose. Tyrode's solution was supplemented with 5 μM blebbistatin, an acto-myosin inhibitor, to remove contractile motion. All images were captured at 1000 frames/s using a CMOS camera (Ultima-L, 1×1 cm 2 field of view with 100×100 pixels, Scimedia, Japan). Fluorescence images of unpaced threads were collected to detect for spontaneous activity and of threads paced with a platinum point stimulation electrode (5 mA current, Harvard Apparatus) at 0.5 Hz increments until MCR is reached. APDs and CVs along the thread were measured at different pacing frequencies with a previously described semi-automated data analysis pipeline.

Optical mapping was used to investigate possible differences in electrical activity of the seeded hiPSC-CMs due to crosslinking of the fibrin threads. No significant differences were found in maximum capture rate (see FIG. 26A-C). A slower conduction velocity was observed in the 20 mg/mL condition when paced at 0.5 Hz and 1 Hz (see FIG. 27A-B). Moreover, an increase in maximum rate change of repolarization action potential duration (APDMxR) was observed in the crosslinked samples with a more prominent change visible in the 20 mg/mL group.

FIG. 26A depicts an activation map of action potential propagation. FIG. 26B depicts data suggesting that no significant differences were seen in maximum capture rate (Control n=3, 2 mg/mL n=3, 20 mg/mL n=3). FIG. 26C depicts data suggesting that representative action potential traces as frequency of electrical pacing increased.

FIG. 27A depicts conduction velocity (Control n=3, 2 mg/mL n=1, 20 mg/mL n=1). FIG. 27B depicts maximum rate repolarization action potential duration (Control n=3, 2 mg/mL n=3, 20 mg/mL n=1).

### Example XIV

### Statistical Analysis

Either a t-test or one-way analysis of variance (ANOVA) with post-hoc Tukey test was used as appropriate. All statistical analyses were performed in GraphPad Prism using a 95% confidence level, and data is presented with * indicating p≤0.05, ** indicating p≤0.01, *** indication p≤0.001, and **** indicating p≤0.0001. Welch's correction for unequal variance was applied.

### Example XV

### Cardiomyocyte Cell-Based Bioelectric Threads

According to one aspect, a mixture of biocompatible support material and electrical impulse transmitting cells is created. The mixture is formed into a thread forming the bioelectric thread having electrical impulse transmitting cells therein. The electrical impulse transmitting cells are in the form of a network of electrically conductive electrical impulse transmitting cells supported by the biocompatible support material.

According to one aspect, methods are provided for treatment of heart defects, such as congenital heart defects, where such defects exhibit poorly coordinated electrical activation of the heart due to a damaged or absent conduction system. Aspects of the present disclosure are directed to use of bioelectric threads as described herein to engineer the cardiac condition system in hearts where conduction between the upper and lower chambers is missing, called atrioventricular (AV) block. According to one aspect, a bioelectric thread is created by deriving cardiomyocytes from human induced pluripotent stem cells, seeding these live cells in a hydrogel system for extrusion-based fiber bundle formation, and subjecting the live fiber bundle formations to mechanical strain and electrical point stimulation to increase conduction velocity during culture. Cardiomyocyte cell-based bioelectric threads have appropriate anisotropy of myofilaments and gap junctions to provide functional conductivity.

Extrusion formation of bundles from 18-gauge needles is accomplished with mixtures of hiPSC-CMs (15 million/mL) in 1% alginate, 3 mg/mL collagen type 1, and 1 mg/mL fibrinogen. Polymerization by calcium in culture medium (RPMI with B27 supplement, 10% FBS, 2% Pen-Strep) forms hydrogel-cell bundles.

The cell bundles are placed in a bioreactor to be stretched and paced cultured with point stimulation integrated to a clamp-based bundle-attachment system. Physiological stretch amplitudes of 1 or 10% strain per day (vs control unstretched bundles) are applied.

CV is assessed by optical mapping to record AP and CaT from the hiPSC-CM intrinsic GCaMP fluorescence signal with a high-speed CMOS camera (>2000 fps) with point stimulus. A gap junction enhancer, rotigaptide (300 nM), is perfused during optical mapping to assess the functional state of gap junctions and impact on CV. Immunohistochemistry is used to evaluate myofilament structure, intercalated disc formation by N-cadherin, and gap junction density and localization by connexin-40 semi-quantitative analyses.

According to one aspect, >50% cardiomyocytes are generated with differentiation culture optimization (duration & concentration of BMP4). Stretch increases hiPSC-CM alignment and tissue anisotropy. Electric field pacing or point stimulation is used to initiate propagating APs and increase intercalated disk formation to polarize CMs. Increasing tension and/or lowering pulse amplitude may increase gap junction density, polarization, and CV, which may be maximized by rotigaptide to reach high physiological CV (1-2 m/s).

The cardiomyocyte cell-based bioelectric threads are inserted into heart muscle tissue using the needle-based delivery device and method described herein and provide function and integration of the tissue-engineered cardiomyocyte bundles to restore AV conduction by improving the cardiac conduction system in localized defects in the cardiac conduction system which may be caused by failure of conduction path formation during development or scar deposition after repair, such as with inert polymer patches. Exemplary cardiomyocyte bundles create electrical bridges exhibiting rapid conduction velocity (0.5-1 m/s) to enable AV electrical coordination in host hearts. According to one aspect, ventricular hiPSC-derived cardiomyocytes (hiPSC-CMs) in bundles exhibit an electrical syncytium and transmit action potential (AP) signals and calcium transients (CaT) across at least 1.5 cm at 2.7 cm/s conduction velocity (CV) between two engineered cardiac tissues. Accordingly, bioelectric threads as described herein provide conduction bridges for direct cellular coupling of cardiomyocytes in vivo.

Engineered cardiac bundles have increased conduction velocity (CV) versus engineered cardiac tissues (ECTs) in vitro. An alginate-collagen-fibrinogen hydrogel-and-CM mixture is extruded by needle and crosslinked by calcium to form cardiac bundles from ventricular GCaMP-expressing hiPSC-CMs. Bundles have cardiac myofilaments and connexin-43-positive gap junctions throughout. In vitro AP duration is 396±33 ms (not shown) and CV is 2.74±0.31 cm/s (n=12), which is at the low end of the physiological range for ventricular conduction. When bundles were placed in contact with two hiPSC-CM ECTs, electrical coupling between bundles and ECTs appeared after 1 day in vitro. FIG. 28A depicts needle-extruded bundles of ventricular hiPSC-CMs in an alginate-collagen-fibrinogen hydrogel pinned into PDMS substrates at static length. FIG. 28B depicts that uniform cells are visible and CMs have active GCaMP Ca2+ signal. Immunofluorescence images of non-stretched bundles show minimal striations with moderate alignment by z-disc labeling (α-actinin) and punctate and disperse connexin-43+ gap junctions. FIG. 28C depicts optical mapping results showing propagation of voltage from a point stimulus (lightning bolt) in a free-floating fiber (no tension) with physiological frequency response. FIG. 28D depicts data showing that ventricular bundles exhibit faster conduction velocity (CV) than engineered cardiac tissues (ECTs) from the same batch of hiPSC-CMs.

### Example XVI

### Coupling of Bioelectric Threads Between Two Tissue Locations

According to one aspect, a bioelectric thread is coupled between two tissue locations. Two parallel linear engineered cardiac tissues (ECTs) were constructed by mixing 95% hiPSC-CMs with 5% hCFs in 1.25 mg/mL rat tail type I collagen (Advanced BioMatrix, Carlsbad, CA, USA) for a total density of 15 million cells per mL. The cell-hydrogen mixture was pipetted onto custom designed, laser-etched PDMS H-molds and allowed to gel at 37° C. for 30-45 minutes, after which ECTs were cultured for 1 week under 1 Hz electrical field pacing in either standard RPMI media or maturation media, with media changes every 2 days. Linear ECTs compact over the 7 days of culture to form an electrical syncytium and began beating between day 3-4 post tissue formation. As shown in FIG. 29, to establish in vitro coupling, blunt forceps were used to gently position the electromechanically active bioelectric thread between two parallel ECTs, ensuring firm contact between the threads and ECTs. Fused bioelectric threads and ECTs were closely monitored for electrical coupling under 1 Hz field electrical stimulation. Electrical coupling was observed within 24 hours, with coupling becoming more robust over 3 days, enabling directed electrical propagation from one linear ECT to the opposite ECT. FIG. 30 demonstrates the propagation of action potential (AP) waveforms at 6 unique locations along the fused tissue, with point stimulation initiated at the bottom section of the left ECT. Healthy 1:1 coupling persisted up to 1.4 Hz, after which 1:2 coupling block is observed at 1.5 Hz between the left ECT and the bioelectric thread due to slow, delayed activation of the thread AP. Taken together, these in vitro findings highlight the feasibility of employing bioelectric threads to establish and direct cardiac conduction pathways in vivo.

### Example XVII

### Core-Shell Bioelectric Threads

According to one aspect, bioelectric threads in the form of a core-shell are contemplated. The core includes biocompatible support material and the shell includes a matrix material. Electrical impulse transmitting cells may be within the core or within the shell or both. Methods of making core-shell embodiments are known to those of skill and include co-extrusion methods to co-extrude a longitudinal core surrounded by a shell. According to one aspect, core-shell bioprinting utilizes two concentric needles to coaxially print distinct “core” and “shell” materials. According to one aspect, cells are included in the core surrounded by a protective acellular shell or an acellular fibrin core is surrounded by the cell-laden shell.

Core-shell bioprinting is performed using a coaxial printhead on a 3D bioprinter fitted with two concentric needles fed by distinct material cartridges. Printing is performed with a 25-37° C. cartridge incubation temperature and 4-25° C. base plate temperature. The core needle diameter ranges from 20-26 G, while the shell needle ranges from 14-20 G. The core and shell materials are independently prepared as described previously herein from biocompatible material. The core material may include electrical impulse transmitting cells. The shell material may include electrical impulse transmitting cells. The core and shell material may include electrical impulse transmitting cells. The core and shell material may be the same or different. Each material is homogenized and loaded into its respective cartridge, then inserted in the print head. Both materials are coaxially printed through two concentric needles using pneumatic control. The shell material is printed using a pressure of 25-90 kPa. The core material is printed using a pressure of 5-70 kPa. A 1-5 cm line is coaxially printed at a feed rate (printing speed) of 1-5 mm/s. Printing is performed either onto a sterile glass surface or into a polydimethylsiloxane (PDMS) mold.

### Example XVIII

### Extrusion Bioprinting

According to one aspect, bioelectric threads formed from extrusion bioprinting methods are contemplated. Extrusion bioprinting is performed using a compatible bioprinter such as a RegenHu 3D Discovery bioprinter, set to a 25-37° C. incubation temperature, 4-25° C. base plate temperature, and 18-28G printing needle. The cell-hydrogel mixture is homogenized and loaded into a print cartridge and a 1-5 cm line is printed at a printing pressure of 5-80 kPa and a feed rate of 1-3 mm/s. Printing is performed either onto a sterile glass surface or into a polydimethylsiloxane (PDMS) mold. Other bioprinters having a controlled pressure driver applied to the cartridge and translating motor moving a needle/print head are useful in the present disclosure.

According to the present disclosure, cell-laden material as described herein is loaded into a syringe and extruded using a bioprinter, as opposed to a manual syringe as described herein. Bioprinters are useful to facilitate future large-scale, consistent biomanufacturing, using mixtures of cells and substrate materials as described herein.

1. An electrically conductive biological device comprising
a biocompatible support material, and
electrical impulse transmitting cells adhered to the surface of the biocompatible support material or contained within the biocompatible support material as a matrix, wherein the biocompatible support material is optionally crosslinked, and
wherein the electrical impulse transmitting cells are in the form of a network of electrically conductive electrical impulse transmitting cells supported by the biocompatible support material.

2. The electrically conductive biological device of claim 1 wherein the electrical impulse transmitting cells comprise cardiomyocytes, cardiac conduction system cells, nodal cells, nerve cells, and/or muscle cells.

3. The electrically conductive biological device of claim 2 wherein the cardiomyocytes comprise human induced pluripotent stem cell-derived cardiomyocytes.

4. The electrically conductive biological device of claim 3 further comprising one or more additional cell types.

5. The electrically conductive biological device of claim 4 wherein the one or more additional cell types comprise cardiac fibroblasts, epicardial cells, immune cells, endothelial cells, smooth muscle cells, vascular stromal cells, and/or pericytes.

6. The electrically conductive biological device of claim 1 in the form of a thread, suture, string, fiber, strip or patch.

7. The electrically conductive biological device of claim 1 wherein the biocompatible support material comprises fibrinogen, fibrin, collagen, gelatin, alginate, hyaluronic acid, chitosan, polyethylene glycol, polylactic acid, polylactic-co-glycolic acid, polycaprolactone or decellularized extracellular matrix.

8. The electrically conductive biological device of claim 1 wherein the biocompatible support material is biocompatible and/or biodegradable.

9. The electrically conductive biological device of claim 1 wherein the biocompatible support material is crosslinked by gelation or a crosslinker.

10. The electrically conductive biological device of claim 1 wherein the crosslinker comprises transglutaminase, thrombin, tannic acid, genipen, citric acid, or phytic acid.

11. The electrically conductive biological device of claim 2 wherein the human induced pluripotent stem cell-derived cardiomyocytes comprise ventricular cardiomyocytes, atrial cardiomyocytes, sinoatrial node cardiomyocytes, atrioventricular node cardiomyocytes, bundle of His cardiomyocytes, bundle branch cardiomyocytes or Purkinje cardiomyocytes.

12. The electrically conductive biological device of claim 1 comprising a crosslinked biocompatible support material lacking electrical impulse transmitting cells therein and having electrical impulse transmitting cells adhered and/or contacted to the surface or embedded in their own biocompatible support material and around the crosslinked biocompatible support material.

13. The electrically conductive biological device of claim 1 comprising electrical impulse transmitting cells contained within the biocompatible support material as a matrix.

14. The electrically conductive biological device of claim 1 comprising electrical impulse transmitting cells contained within the biocompatible support material as a matrix and surrounded by cell-free biocompatible support material.

15. The electrically conductive biological device of claim 1 comprising human induced pluripotent stem cell-derived cardiomyocytes of at least 70% purity and further comprising less than or equal to 25% human cardiac fibroblasts.

16. The electrically conductive biological device of claim 1 further cultured with an antifibrinolytic agent, a degradation modulating agent, and/or a viability enhancing agent.

17. The electrically conductive biological device of claim 1 in the form of a thread and having an end portion with or without cells for facilitating delivery to tissue.

18. A method of making the electrically conductive biological device of claim 1 comprising
culturing a combination of the biocompatible support material and the electrical impulse transmitting cells to form a network of electrically conductive electrical impulse transmitting cells supported by the biocompatible support material.

19. (canceled)

20. (canceled)

21. The method of claim 18 wherein the combination of the biocompatible support material and electrical impulse transmitting cells is mechanically stretched during culturing.

22. The method of claim 18 wherein the combination of the biocompatible support material and electrical impulse transmitting cells is subject to electrical field or point stimulation during culturing.

23. The method of claim 18 wherein the combination of the biocompatible support material and electrical impulse transmitting cells is subject to metabolic and/or maturation stimulating conditions.

24. The method of claim 18 wherein the biocompatible support material and electrical impulse transmitting cells are mixed together and the mixture is formed into a thread such as by extrusion manually or with a bioprinter which is subject to culture conditions to form a network of electrically conductive electrical impulse transmitting cells within the biocompatible support material.

25. The method of claim 18 wherein the biocompatible support material is formed into a thread, optionally crosslinked, and the crosslinked biocompatible support material in the form of a thread is adhered, surrounded, and/or contacted with the electrical impulse transmitting cells under culture conditions to form a network of electrically conductive electrical impulse transmitting cells on the surface of the crosslinked biocompatible support material.

26. The method of claim 18 wherein the biocompatible support material alone and mixed with the electrical impulse transmitting cells are co-extruded manually or with bioprinting in a co-axial core-shell arrangement in the form of a thread and optionally crosslinked and under culture conditions to form a network of electrically conductive electrical impulse transmitting cells in the outer ring or shell of the thread.

27. The method of claim 18 wherein the biocompatible support material alone and mixed with the electrical impulse transmitting cells are co-extruded manually or with bioprinting in a co-axial core-shell arrangement in the form of a thread and optionally crosslinked and under culture conditions to form a network of electrically conductive electrical impulse transmitting cells in the inner core of the thread surrounded by a protective and/or insulating shell of biocompatible support material.

28. The method of claim 18 wherein the electrically conductive biological device exhibits a conduction velocity greater than 2 cm/s and a maximum capture rate greater than 1 Hz.

29. The method of claim 18, wherein the combination further includes one or more of cardiac fibroblasts, epicardial cells, immune cells, endothelial cells, smooth muscle cells, vascular stromal cells, and/or pericytes.

30. A method of repairing cardiac conduction defects in a subject in need thereof comprising
implanting the electrically conductive biological device of claim 1 between two target cardiac locations, wherein the electrically conducting device comprises human induced pluripotent stem cell-derived cardiomyocytes and cardiac fibroblasts as a network of electrically conductive electrical impulse transmitting cells supported by the biocompatible support material,
whereby the electrically conductive biological device establishes a continuous electrically conductive pathway between the two target cardiac locations to facilitate synchronized impulse propagation and/or to reduce arrhythmia risk.

31. A combination comprising
a needle having an end portion for insertion within tissue and having the electrically conductive biological device of claim 1 within the needle, optionally the electrically conductive biological device is positioned within a tube which is positioned within the needle.

32. The combination of claim 31 further comprising a lubricant contacting the electrically conductive biological device.

33. (canceled)

34. The combination of claim 31 wherein the electrically conductive biological device includes an exposed anchor, end or tail portion which anchors the electrically conductive biological device within tissue.

35.-37. (canceled)

38. The combination of claim 31 further comprising a retraction mechanism for retracting the needle and exposing the electrically conductive biological device.

39. The combination of claim 31 wherein the optional tube includes two longitudinal and separable portions proximal to the end portion of the needle.

40. The combination of claim 31 wherein the needle comprises two longitudinal, interlocking, and separable portions between which the electrically conductive biological device is positioned.

41. The combination of claim 31 further comprising a lubricant on the surface of the electrically conductive biological device.

42. The combination of claim 31 wherein the needle includes an inner surface of a friction reducing material or surface modification.

43. The combination of claim 31 wherein the optional tube includes an inner surface of a friction reducing material or surface modification.

44. A method of delivering the electrically conductive biological device of claim 1 comprising
inserting the electrically conductive biological device between two longitudinal and separable portions of a needle, wherein the electrically conductive biological device is optionally within a tube,
inserting the needle into tissue,
withdrawing the needle and optional tube together or individually with the electrically conductive biological device remaining within the tissue.

45.-50. (canceled)
